(19) United States
(12) Patent Application Publication
(10) Pub. No.: US 2015/0167014 A1
(43) Pub. Date: Jun. 18, 2015

(54) MODULATION OF PLANT GROWTH BY VAS1 MUTATION

(71) Applicant: THE SALK INSTITUTE FOR BIOLOGICAL STUDIES, La Jolla, CA (US)

(72) Inventors: Joseph Noel, San Diego, CA (US); Joanne Chory, Del Mar, CA (US); Yongxia Guo, San Diego, CA (US); Zuyu Zheng, San Diego, CA (US)

(73) Assignee: THE SALK INSTITUTE FOR BIOLOGICAL STUDIES, La Jolla, CA (US)

(21) Appl. No.: 14/560,908

(22) Filed: Dec. 4, 2014

Related U.S. Application Data
(60) Provisional application No. 62/028,124, filed on Jul. 23, 2014.
(60) Provisional application No. 61/912,398, filed on Dec. 5, 2013.

(51) Int. Cl. C12N 15/82; C12N 9/00

(52) U.S. Cl. CPC C12N 15/8261; C12N 9/93

(57) ABSTRACT

The present invention relates to mutants of the plant aminotransferase VAS1 and to transgenic plants expressing said mutant protein that exhibit a modulated response to shade. In certain embodiments, the mutations of VAS1 uncouple VAS1 metabolic coordination of auxin and ethylene homeostasis, so that reduction of auxin is no longer linked (via VAS1) to reduction of ethylene. Such uncoupling allows for the generation of plants carrying a VAS1 mutant transgene that demonstrate a modulated response to shade, for example, less hypocotyl growth.

### CROSS REFERENCE TO RELATED APPLICATIONS

This application claims priority to U.S. Provisional Patent Application No. 61/912,398, filed on Dec. 5, 2013; and claims priority to U.S. Provisional Patent Application No. 62/028,124, filed on Jul. 23, 2014, priority to each of which is claimed, and the contents of each of which are hereby incorporated by reference in their entireties.

### GRANT INFORMATION

This invention was made with government support under Grant No. 5R01GM52413 awarded by the National Institutes of Health and Grant Nos. EEC-0813570 and MCB-0645794 awarded by the National Science Foundation. The government has certain rights in the invention

### SEQUENCE LISTING

The instant application contains a Sequence Listing which has been submitted electronically in ASCII format and is hereby incorporated by reference in its entirety. Said ASCII copy, created on Feb. 18, 2015, is named 082700.0121_SL.txt and is 82,978 bytes in size.

### 1. INTRODUCTION

The present invention relates to mutants of the plant aminotransferase VAS1 and to transgenic plants expressing said mutant protein that exhibit a modulated response to shade.

### 2. BACKGROUND OF THE INVENTION

Organism growth and development is complexly and elegantly regulated by the interactions of the environmental signals with endogenous growth programs. Many of the diverse extrinsic and intrinsic cues converge on hormone regulation^{1,2,4}. For instance, confronting shade (e.g., where the ratio of red light to far-red light is smaller than 1), plants promptly increase the biosynthesis of two classical hormones, auxin and ethylene, resulting in rapid elongation growth of hypocotyls and petioles, respectivel^{10,16,17}. Such exaggerated elongation growth allows plants to outcompete their neighbors for energy source sunlight, representing a fundamental adaptive strategy for the sessile plants^{16,17}.

In agriculture, where it is typically desirable to increase crop yield, plants may be grown at high density, creating shade on other plants. The shaded plants may then exhibit a shade avoidance response (“SAR”), including, as mentioned above, increased hypocotyl and petiole length, and/or one or more of decreased biomass, decreased chlorophyll, altered flowering time, lower seed yield and/or poorer seed quality. In addition, plants exhibiting these characteristics are more vulnerable to herbivores and insect pests. These manifestations of the SAR would also be detrimental to decorative plants. Accordingly, it would be desirable to modulate one or more aspect of the SAR in certain plants.

### 3. SUMMARY OF THE INVENTION

The present invention relates to mutants of the plant aminotransferase VAS1 and to transgenic plants expressing said mutant protein that exhibit a modulated response to shade. It is based, at least in part, on the discoveries that (i) VAS1 metabolically coordinates the homeostasis of auxin and ethylene and functions as a metabolic rheostat that concurrently reduces the amount of auxin and ethylene for preventing plants from over-reacting to shade (FIG. 1A) and (ii) mutation of VAS1 was found to uncouple these pathways, so that reduction of auxin was no longer linked (via VAS1) to reduction of ethylene. Such uncoupling allows for the generation of plants carrying a VAS1 mutant transgene that demonstrate a modulated response to shade. For example a transgenic VAS1 mutant plant may, in response to shade and relative to a control plant lacking the transgene, exhibit less hypocotyl growth (shorter hypocotyl(s)) but similar petiole elongation.

### 4. BRIEF DESCRIPTION OF THE FIGURES

FIG. 1A-C. VAS1's function and three-dimensional structure of VAS1. (A) Model for VAS1 metabolic regulation of the homeostasis of auxin and ethylene to modulate the elongation of hypocotyl and petiole, respectively. The petiole of the first set of true leaves and hypocotyl are highlighted with yellow dotted line (top) and red dotted line (bottom), respectively. SAM, S-adenosyl-L-methionine; MTA, methylthioadenosine; MTR, methylthioribose; MTR-1-P, methylthioribose phosphate. (B) Ribbon diagram of the VAS1 structure bound to PLP and KMBA. The internal Schiff base between Lys 233 and PLP, KMBA, Met 19, Ile 267, Arg 241 and Arg 362 are shown as sticks. The negative charge of PLP phosphate group is well balanced with the positive charge of Arg 241. Arg 362 tethered the distal carboxylate of KMBA. The hydrophobic thioether chain of KMBA is interacting with Ile 267 and Met 19. (C) Ribbon diagram of the VAS1 structure bound to PLP and 3-IPA analogue IAA. The internal schiff base between Lys 233 and PLP, KMBA, Met 19, Ile 267, Arg 241 and Arg 362 are shown as sticks. Arg 362 tethered the distal carboxylate of IAA. The indole ring of IAA is interacting with Ile 267 and Met 19. The chemical structure of Met, KMBA, IPA and IAA are shown alongside. The simulated annealing omit 2FO-FC electron density map of KMBA and IAA are shown (contoured at 1.0 σ).

FIG. 2A-F. Directly evolved VAS1 I267M decouples VAS1's dual roles in modulating the auxin and ethylene metabolism in vitro. (A) Structural superposition of the VAS1•PLP•KMBA complex and VAS1•PLP•IAA complex. The PLP-Lys 233 Schiff base, Ile 267, Met 19, KMBA and IAA are shown as sticks. (B) The relative enzymatic activities of VAS1 and its evolved variants using Met and 3-IPA as the substrates. (C) Met is the most suitable amino donor in the VAS1 catalyzed transamination reaction using 3-IPA as the amino acceptor. (D) His is the most efficient amino donor for the VAS1 I267M using 3-IPA as the amino acceptor. (E,F) Model for the biochemical functions of VAS1 (E) and VAS1 I267M mutant (F). The dotted arrow lines indicate that VAS1 I267M couldn't regulate these reactions.

FIG. 3A-E. In planta, VAS1 I267M maintains VAS1's role in regulating auxin metabolism, but loses VAS1's control of ethylene metabolism. (A) VAS1 I267M transgene decreases the length of hypocotyls, but not petioles, of vas1-2 sav3-1 double mutant. The plants were grown on ½ MS plates, and kept under white light condition (Wc) for 6 d and then remained in Wc for 4 d (−) or transferred to shade for 4 d (+). The table below the plant pictures briefly summarizes the correlation between auxin level and hypocotyl length, between ACC level and petiole length, in various mutants/transgenic lines grown under shade (+). In the table, the symbol “−” indicates the levels of IAA and ACC are relatively low or the hypocotyls and petioles are relatively short; while the symbol “↑” indicates the levels of IAA and ACC are relatively high or the hypocotyls and petioles are relatively long. Detailed quantifications are shown in B-E. (B) VAS1 I267M transgene decreases the hypocotyl length of vas1-2 sav3-1 double mutant (n=12). Two independent 35S::VAS1(I267M)-YFP transgenic lines in vas1-2 sav3-1 double mutant backgrounds are shown. (C) VAS1 I267M transgene doesn't affect the petiole length of vas1-2 sav3-1 double mutant (n=28). The petiole length of the first set of true leaves are measured. (D) VAS1 I267M transgene decreases the auxin level of vas1-2 sav3-1 double mutant (n=4). (E) VAS1 I267M transgene doesn't affect the ethylene level of vas1-2 sav3-1 double mutant (n=4). Results are shown as mean±s.e.m. ***P<0.001 (two-tailed Student's t-test). The comparison is made between wild-type Col plants and mutants under the same growth conditions and same treatment.

FIG. 4. The overall structure of VAS1 and a magnified view of PLP binding to the VAS1 active site. VAS1 functioned as dimer, Lys 233ε-amino group made an external Schiff base with PLP. PLP is shown as color-coded sphere (overall structure) and sticks (magnified view) where carbon is orange, nitrogen is blue and oxygen is red. Catalytic site residue K233 is shown as color-coded sphere (overall structure) and sticks (magnified view) where carbon is green and nitrogen is blue. PLP form the “internal aldimine” with K233 and have a strong interaction with Asn 176 (interact with the phenoic oxygen O3 of PLP), Asp204 (interact with the pyridine nitrogen of the cofactor PLP) and Arg241 (balanced the negative charge of the phosphate group). PLP-K233, Asn 176, Asp204 and Arg241 are shown as sticks.

FIG. 5. Ribbon diagram of the VAS1 monomer in a complex with PLP and KMBA. The helices (α1-α18) and strands (β1-β9) are indicated. Large domain strands are arranged as β1↑-β7↓-β6↑-β5↑-β4↑-β2↑-β3↑, N-terminal and C-terminal small domain are shown in gold and green, respectively. Large domain is colored blue. PLP and KMBA are shown as sticks.

FIG. 6. Ribbon diagram of the VAS1 structure bound to PLP and KMBA. The catalytic Lys 233, PLP, KMBA, Met19 and Ile267 are shown as van der Waals spheres where oxygen is red and sulfur is yellow and carbons are colored green (lys233 and Met19), orange (PLP) and white (KMBA and 1267).

FIG. 7. Ribbon diagram of the VAS1 structure bound to PLP and IPA analog IAA. The catalytic Lys 233, PLP, IAA, Met19 and Ile267 are shown as van der Waals spheres where oxygen is red, nitrogen is blue and sulfur is yellow and carbons are colored green (lys233 and Met19), orange (PLP) and white (IAA and 1267).

FIG. 8. IPA is the best amino acceptor for I267M mutant. The ketoacids, including glyoxylate, pyruvate, 2-ketobutyrate, KMBA, 2-oxoglutarate, and oxaloacetate could not function as the amino acceptors of the I267M. The relative activity is very low using phenylpyruvate and 4-hydroxyphenylpyruvate as co-substrate.

FIG. 9A-B. Steady-state kinetic analyses of VAS1 I267M. Curves were fit to the Michaelis-Menten equation and are displayed from left to right. Fixed concentration of L-His (10 mM) and variable concentrations of 3-IPA from 10 to 200 μM (A) Fixed concentration of 3-IPA (300 μM) and variable concentrations of L-His from 0.1 to 10 mM (B). Apparent KM, Vmax and kcat of VAS1 are shown above each curve and standard errors calculated from Graphpad Prism 5 software (www.graphpad.com).

FIG. 10. The amount of VAS1 protein in 35S::VAS1-YFP transgenic lines is similar to that of VAS1(I267M) protein in 35S::VAS1(I267M)-YFP transgenic lines. The 7 day old 35S::VAS1-YFP transgenic lines and 35S::VAS1(I267M)-YFP transgenic lines in vas1-2 sav3-1 mutant background were used. The VAS1 and VAS1(I267M) protein levels were determined by immunoblotting using Anti-GFP antibody. The same samples were probed with Anti-Actin antibody to show the protein loading control.

FIG. 11A-C. Model of VAS1 metabolic linking auxin and ethylene biosynthesis and the three dimensional structure of VAS1. A. The metabolic hub linking auxin and ethylene biosynthesis through VAS1. B. Comparison of the open-closed conformational change of VAS1. C. A magnified view of PLP binding to the VAS1 active site.

FIG. 12A-B. KMBA and IAA share the same substrate binding pocket A. VAS1 binding site is occupied by KMBA. B. VAS1 binding site is occupied by IPA's analogue IAA.

FIG. 13A-F. Structure-based engineering of VAS1 to decouple the auxin and ethylene biosynthetic pathway. A. Structural superposition of the VAS1•PLP•IAA and VAS1•PLP•KMBA complex. B. The relative enzymatic activity of VAS1's substrate binding mutants using Met and IPA as the substrates. C. Met is VAS1's most suitable amino donor using IPA as the cosubstrate. D. His is the best amino donor for VAS1's I267M mutant using IPA as the cosubstrate. E, F Steady-state kinetic analyses of VAS1's I267M mutant.

FIG. 14A-C. Phenotypes of the I267M transgenic lines. A. I267M mutant transgene suppresses the longer hypocotyl, but not longer petiole of vas1 sav3 double mutant. B. Quantification of the hypocotyl length. C. Quantification of the petiole length.

FIG. 15A-I. Amino acid sequences of VAS1 in various plants. (A) Arabidopsis thaliana, NCBI Accession No. NP_{—}178152 (SEQ ID NO: 14); (B) Arabidopsis lyrata subsp. lyrata, NCBI Accession No. XP_{—}002887833 (SEQ ID NO: 15); (C) Ricinus communis, NCBI Accession No. XP_{—}002517536 (SEQ ID NO: 16); (D) Vitis vinifera, NCBI Accession No. XP_{—}002284955 (SEQ ID NO: 17); (E) Vitis vinifera, NCBI Accession No. XP_{—}002284514 (SEQ ID NO: 18); (F) Populus trichocarpa, NCBI Accession No. XP_{—}002299622 (SEQ ID NO: 19); (G) Physcomitrella patis, NCBI Accession No. XP_{—}001753569 (SEQ ID NO: 20); (H) Oryza sativa, NCBI Accession No. NP_{—}001042188 (SEQ ID NO: 21); and (I) Chlamydomonas reinhardtii (SEQ ID NO: 22).

FIG. 16A-B. (A) Aligned amino acid sequences of VAS1 in various plants, including Catharantus roseus (Madagasar rosy periwinkle) (SEQ ID NO: 23), Solanum lycopersicum (tomato) (SEQ ID NO: 24), Populus trichocarpa (black cottonwood) (SEQ ID NO: 25), Ricinus communis (castor oil plant) (SEQ ID NO: 26), Gossypium hirsutum (cotton) (SEQ ID NO: 27), Arabidopsis thaliana (mouse-ear cress) (SEQ ID NO: 28), Zea mays (corn) (SEQ ID NO: 29), Oryza sativa (Asian rice) (SEQ ID NO: 30), Brachypodium distachyon (purple false brome) (SEQ ID NO: 31), Selaginella moellendorfii (SEQ ID NO: 32), and Vitis vinifera (common grape vine) (SEQ ID NO: 33). (B) Alignment at amino acid residues 16, 233 and 267 (SEQ ID NOS 34-72, respectively, in order of appearance).

FIG. 17. VAS1 homolog is conserved through plant lineages.

FIG. 18A-D. Rationale: natural variation in the green plant lineage decouples IPA and Met recognition. (A) Arabidopsis thaliana. (B) Selaginella moellendorfii (C) Physcomitrella patens (D) Chlamydomonas reinhardtii.

### 5. DETAILED DESCRIPTION OF THE INVENTION

For clarity and not by way of limitation, the detailed description is divided into the following subsections:

(i) the VAS1 active site;

(ii) VAS1 mutations;

(iii) plants carrying VAS1 mutations; and

(iv) methods of modulating plant growth.

### 5.1 The VAS1 Active Site

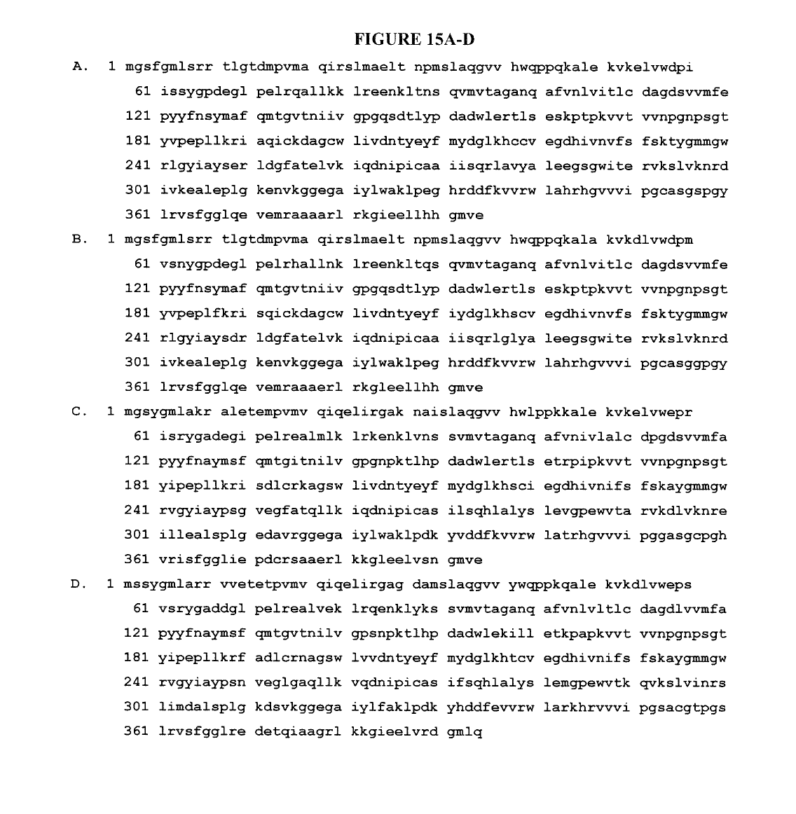

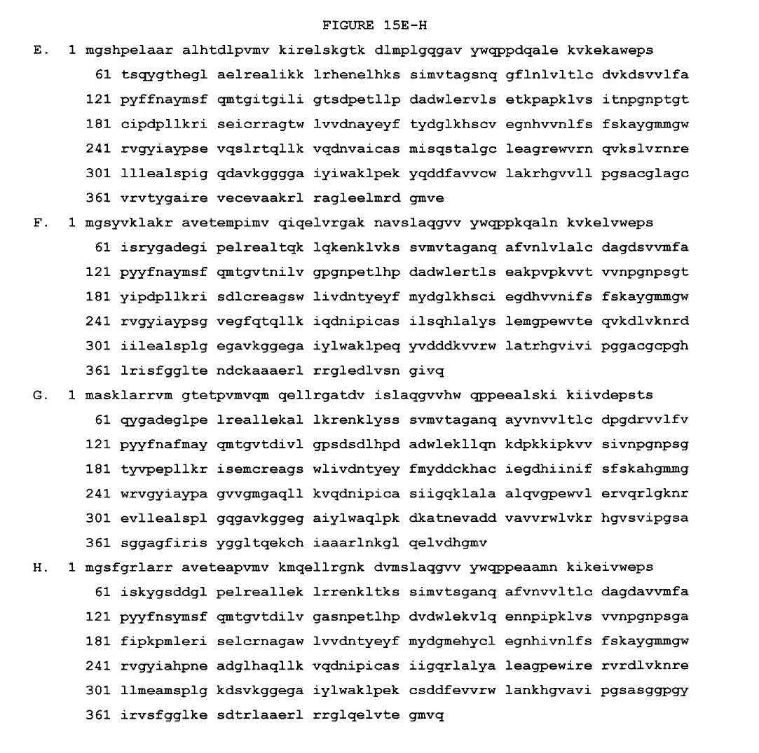

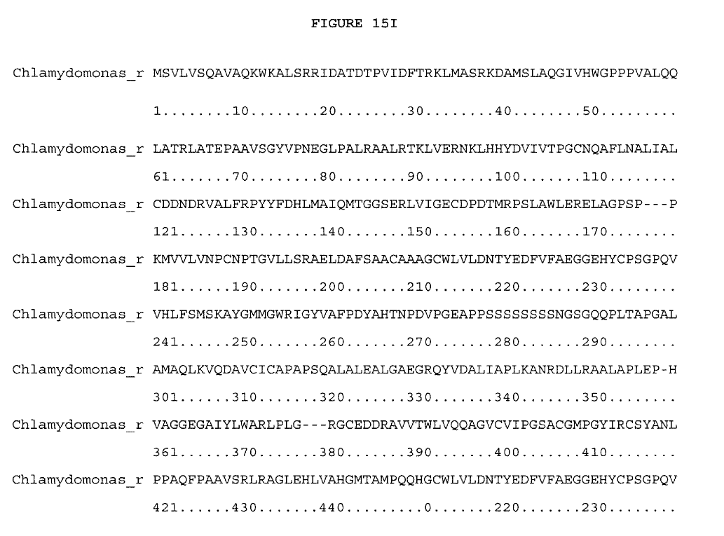

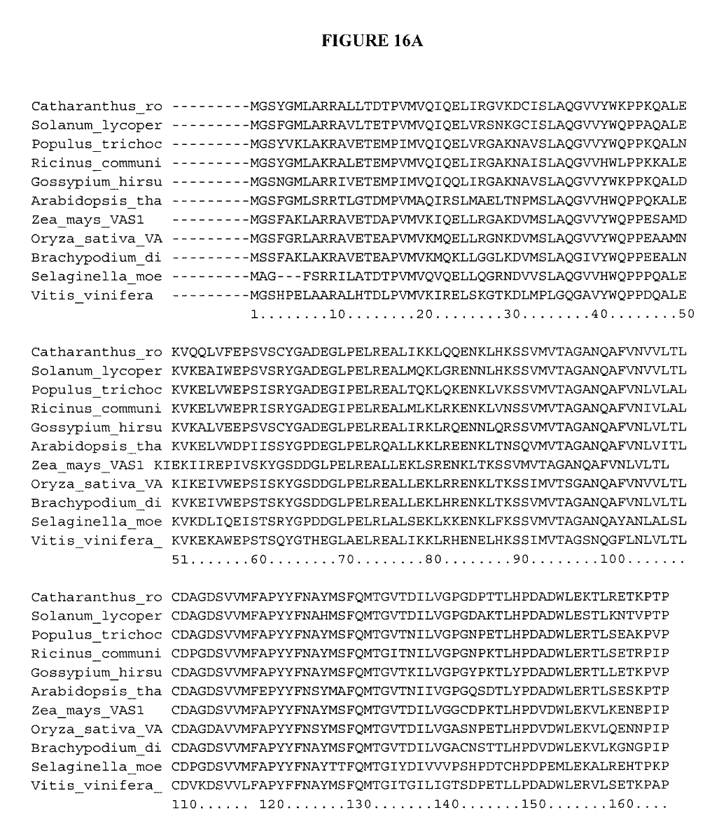

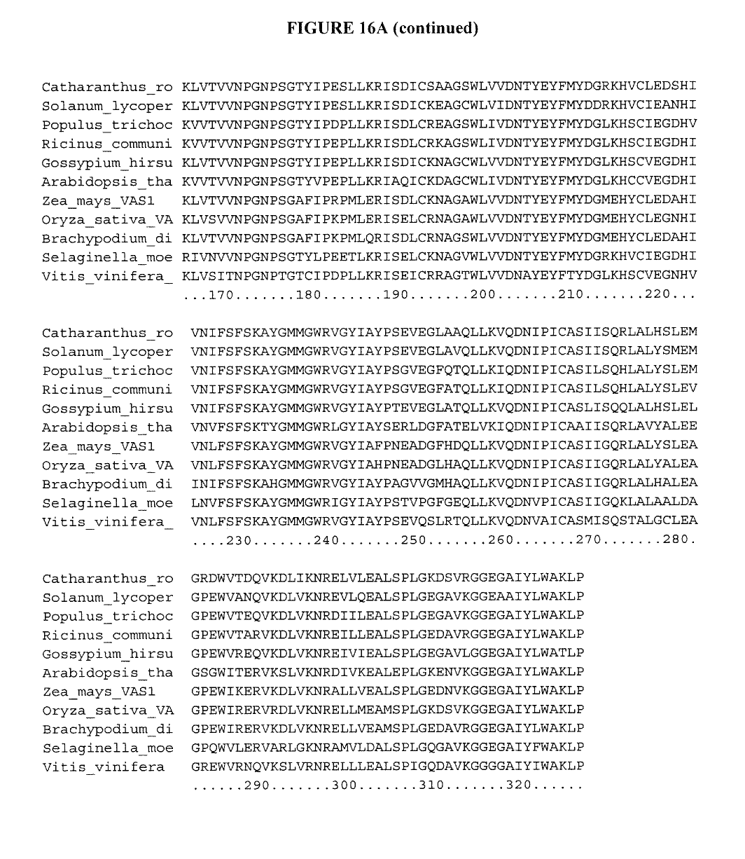

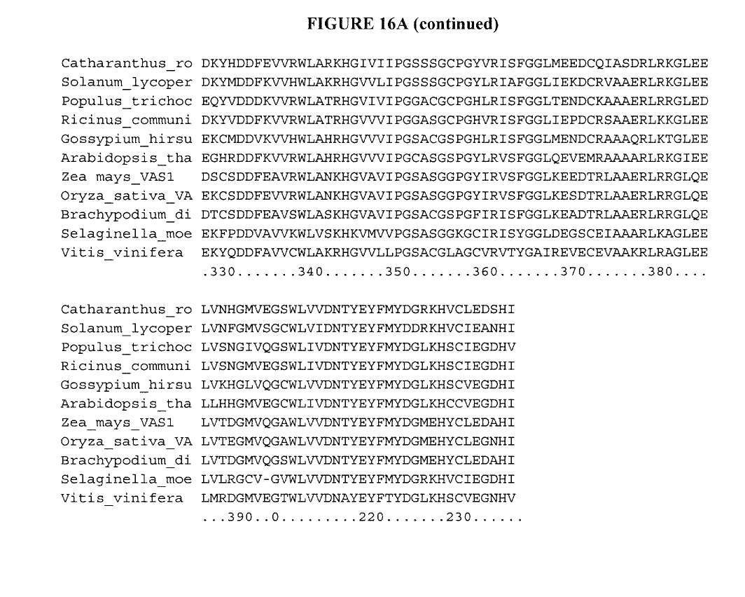

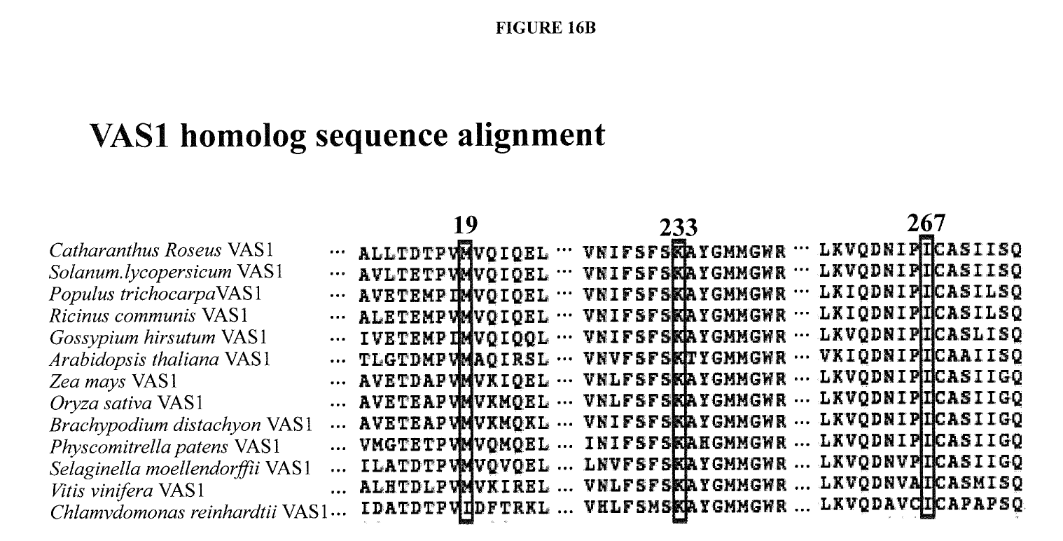

The crystal structure of VAS1 of Arabidopsis thaliana was used as the basis for the following, but, in view of homologies between VAS1 proteins of different plants (see FIG. 15A-H), the skilled artisan would be able to extend the conclusions reached regarding the A. thaliana VAS1 protein to other plant species, including but not limited to those represented in FIG. 15A-I and FIG. 16.

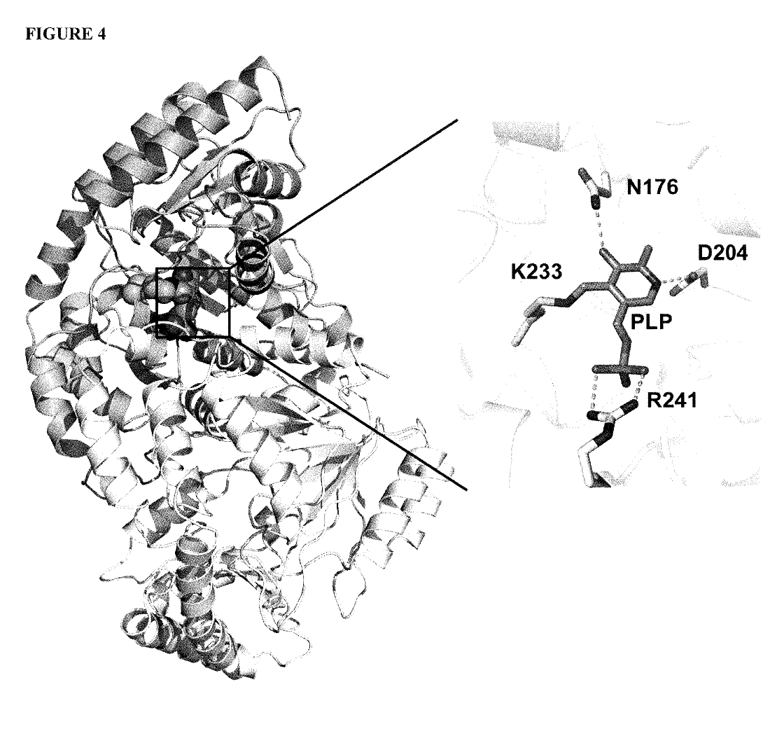

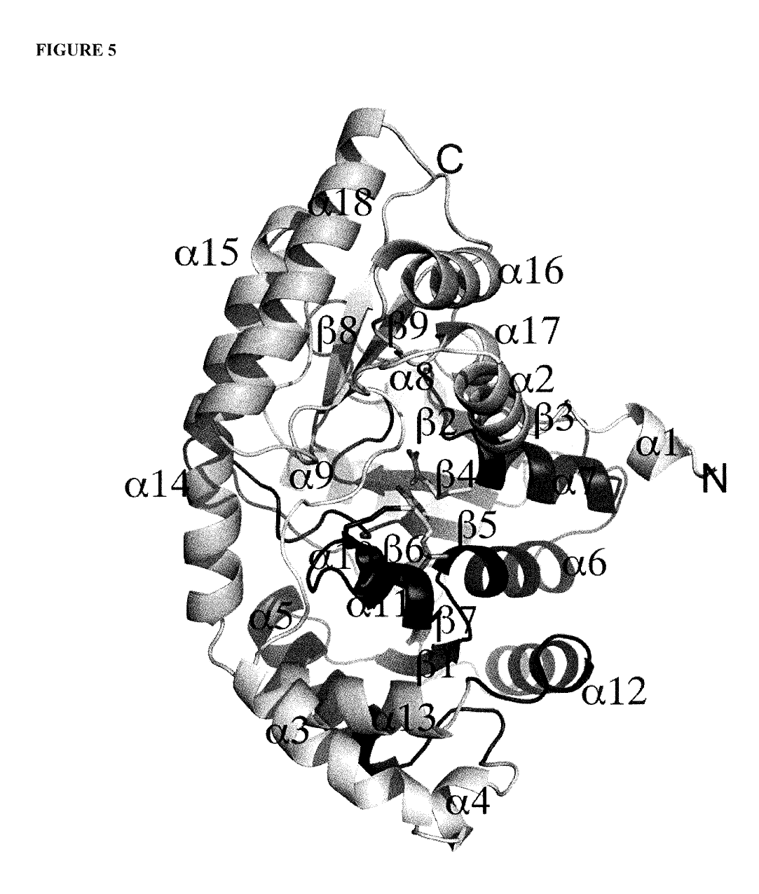

The atomic resolution structure of VAS1•PLP complex showed that VAS1 forms a symmetric dimer, with each monomer containing an active-site pocket around the 2-fold axis (FIG. 4). The active-site pocket is located at the interface of small domain (resi7-65, 270-392, α1-α4, α13-α18, β8-β9) and large domain (resi 66-269, α5-α12,β1-β7) of one monomer (FIG. 4). Helixes α1-4, α6-7 and α12-13 correspond to the dimer interface interaction and the interface area is about 3065 Å² (FIG. 4). The core of the large domain contains the sharply twisted seven-stranded β sheets (the sheets are parallel except β7 arranged as β1↑-β7↓-β6↑-β5↑-β4↑-β2↑-β3↑) that are surrounded by 8 α helixes. The 8 α helixes and 7 β sheets make Oa sandwich architecture (FIG. 4). The cofactor PLP is located at the bottom of the active-site cavity with its si-face directed toward the protein side and serve as an electron sink (FIGS. 4 and 5). PLP forms an internal aldimine bond with the c-amino group of Lys 233 in the large domain and interacts extensively with the residues located at or near one end of the seven-stranded sheet of the large domain. Specifically, Asn 176 and Tyr 207 interact with the phenoic oxygen O3 of PLP with distance of 2.97 A (O3′-NH2) and 2.91 A (03′-OH), respectively (FIG. 4). Asp 204 interacts with the pyridine nitrogen of the cofactor PLP, thus maintaining the cofactor in the protonated form (FIG. 4)¹⁸. The negative charge of the phosphate group of PLP is well balanced with the positive charge of Arg 241 (FIG. 4). Pyridine ring of PLP is sandwiched by the 4-hydroxyphenyl ring of Tyr 123 and hydroxy side-chain of Thr 206 (FIG. 4).

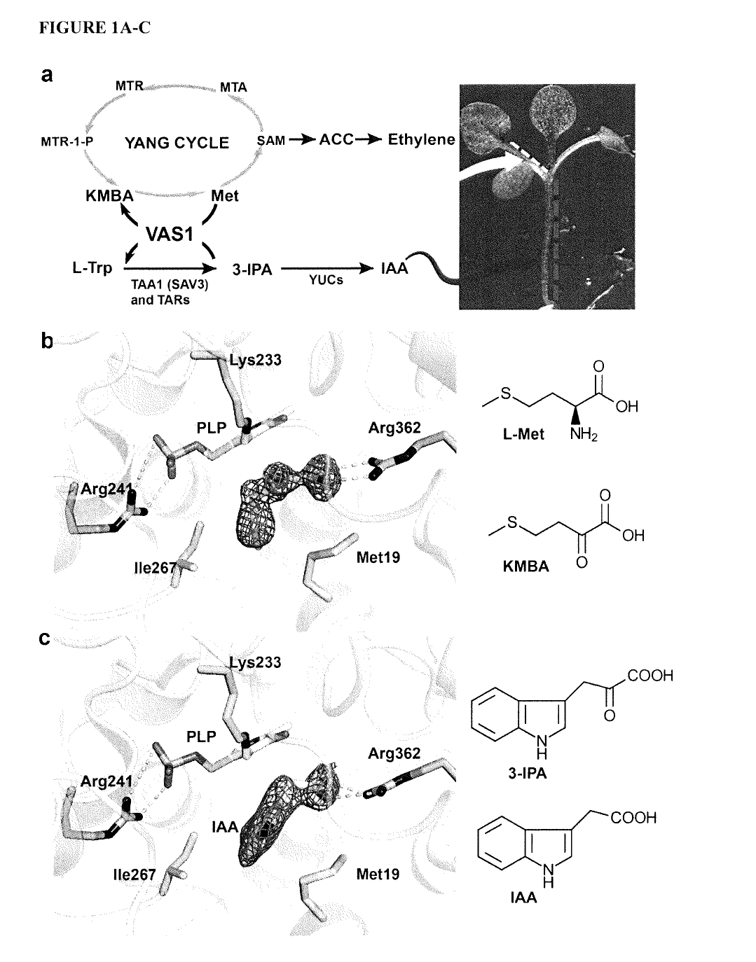

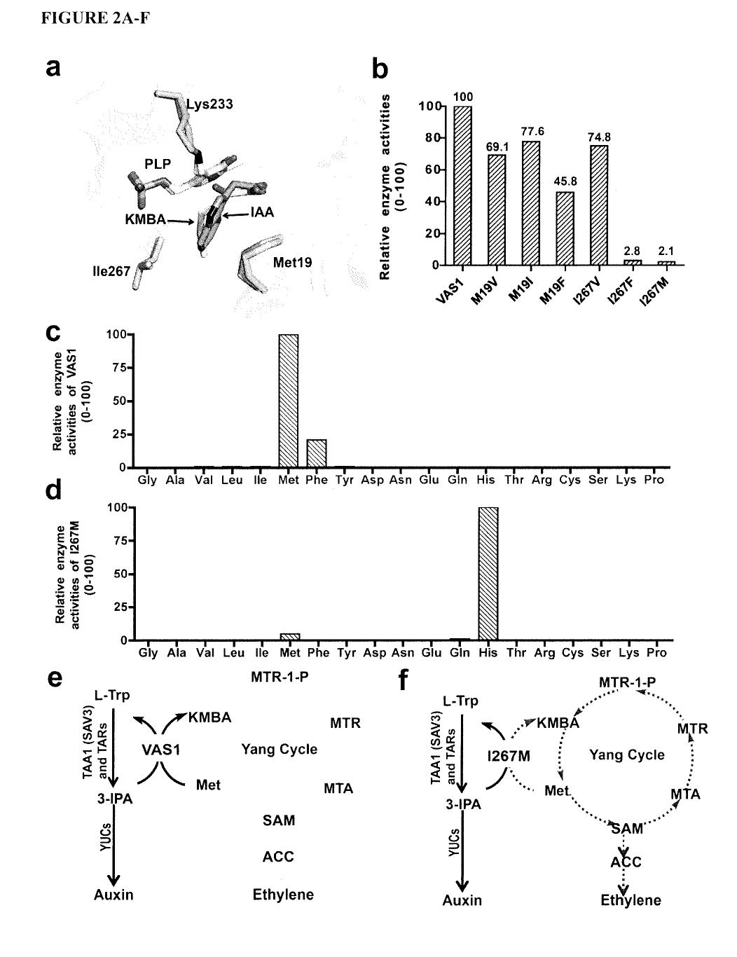

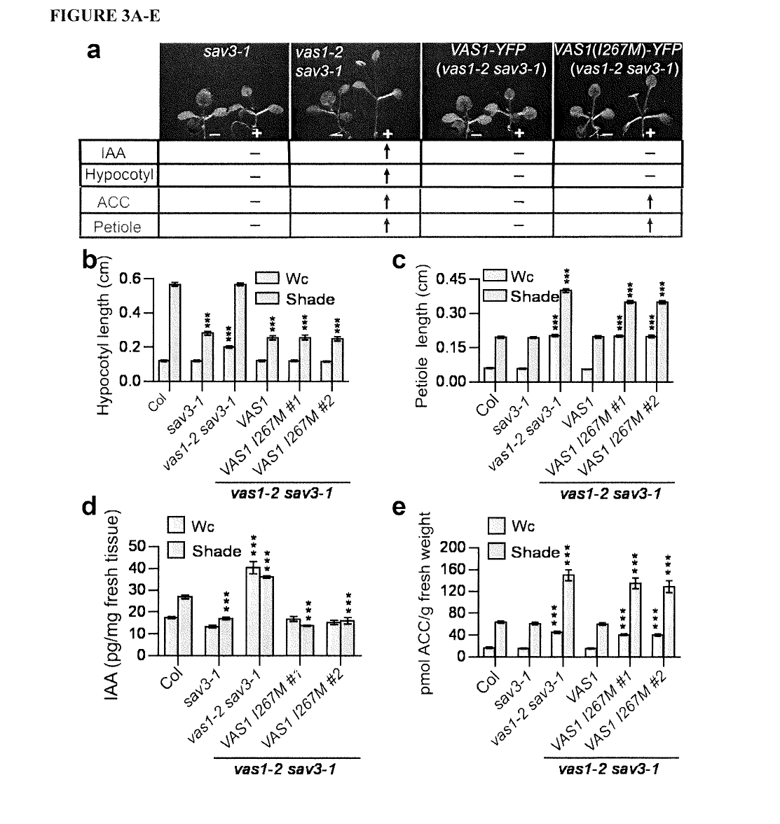

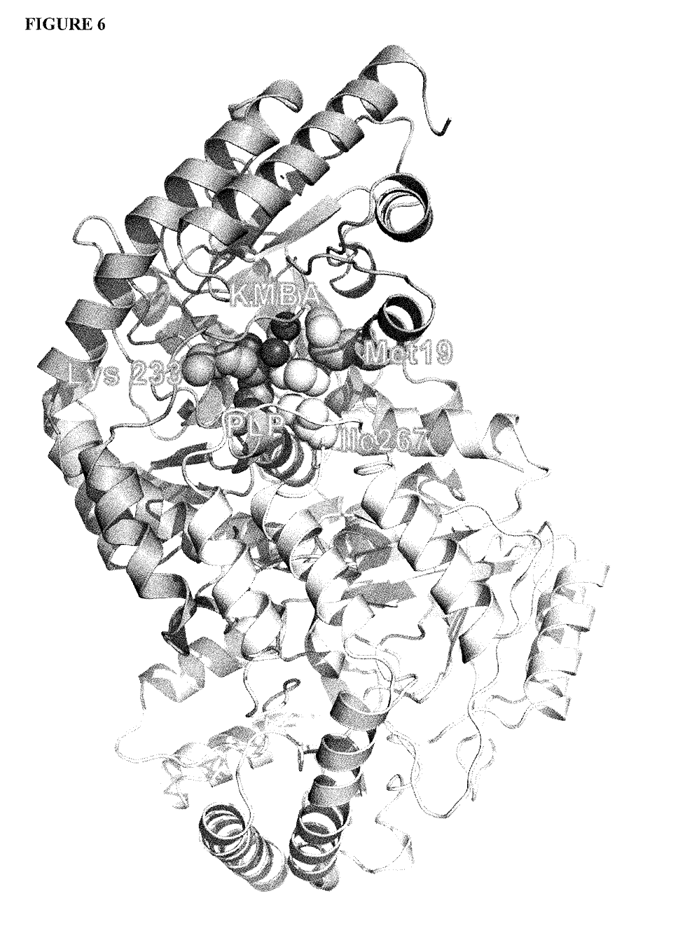

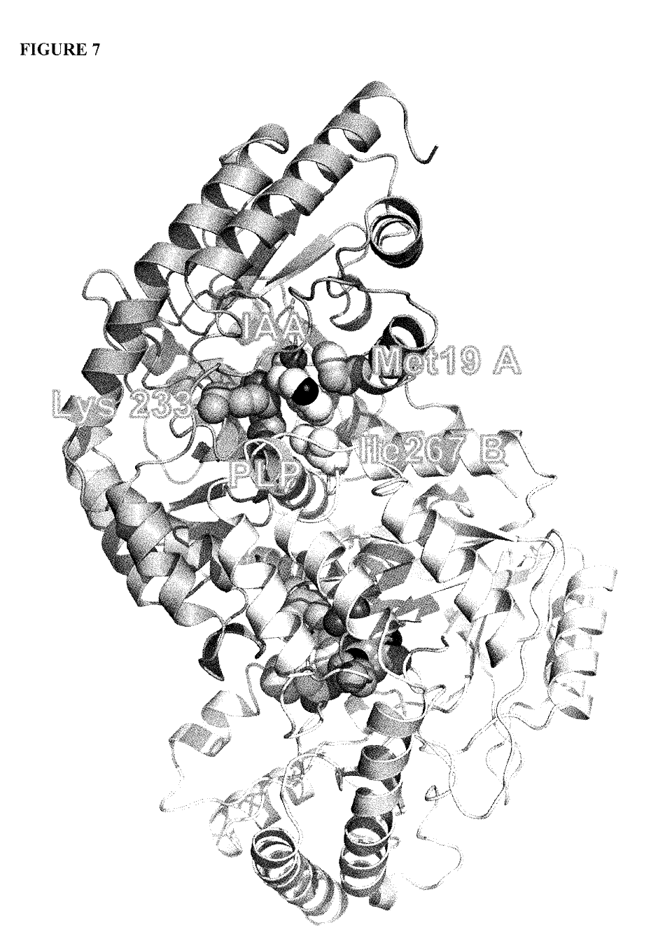

As discussed in the working example below, VAS1 was co-crystallized with the cofactor PLP and either the product KMBA (VAS1•PLP•KMBA) or the substrate 3-IPA analog IAA. In these complexes, cofactor PLP binds to VAS1 in the same way as that in VAS1•PLP structure. Comparison of VAS1•PLP•KMBA to VAS1•PLP•IAA complex showed that, although structurally quite distinct, KMBA and IAA bind to VAS1 in almost the same way (FIG. 2A): both are recognized by Met 19, Ile 267* (asterisk indicates the residue from another subunit of the dimmer unit) and Arg 362 (FIGS. 1B, 1C, 2A, 6 and 7); the conserved Arg 362 guanidine group tethers the carboxylate group of both KMBA and IAA by making salt bridge with an “end-on” geometry (FIGS. 1B, 1C, and 2A); both the thioether moiety of KMBA and the indole ring of IAA formed non polar interactions with the side chains of Met 19 and Ile 267*(FIGS. 1B, 1C, 2A, 6 and 7) (asterisk indicates the residue from another subunit of the dimer). Without being bound by any theory, these results suggest that aminotransferase VAS1 recognizes two substrates Met and 3-IPA by essentially the same interaction network in essentially the same binding pocket.

### 5.2 VAS1 Mutations

In certain non-limiting embodiments, the present invention provides for a mutant VAS1 enzyme having a mutation in the VAS1 active site that results in a modulation of the activity of VAS1, relative to unmutated enzyme, to convert Methionine to KMBA and/or to convert 3-IPA to L-Trp.

In certain non-limiting embodiments, the present invention provides for a mutant VAS1 enzyme having a mutation in the VAS1 active site that results in a reduction of the activity of VAS1, relative to unmutated enzyme, to convert Methionine to KMBA and/or to convert 3-IPA to L-Trp.

In certain non-limiting embodiments, the present invention provides for a mutant VAS1 enzyme having a mutation in the VAS1 active site that, when the mutant VAS1 is expressed in a plant, results in a modulated response of said plant to shade.

In certain non-limiting embodiments, the present invention provides for a mutant VAS1 enzyme having a mutation in the VAS1 active site that, when the mutant VAS1 is expressed in a plant, results in a modulated response of said plant to shade wherein negative regulation of auxin or ethylene by the mutant VAS1 is altered.

In certain non-limiting embodiments, the mutation in the active site is a mutation of an amino acid selected from the group consisting of Met19, Lys233, Ile267 and R362.

In certain non-limiting embodiments, the mutation in the active site is a mutation of an amino acid selected from the group consisting of Met19, Lys233, Ile267, R362 or a combination thereof in a VAS1 enzyme from a plant selected from the group of Arabidopsis thaliana, Arabidopsis lyrata subsp. lyrata, Catharantus roseus (Madagasar rosy periwinkle), Solanum lycopersicum (tomato), Gossypium hirsutum (cotton), Zea mays (corn), Brachypodium distachyon (purple false brome), Selaginella moellendorfii, Ricinus communis (castor oil plant), Vitis vinifera (common grape vine), Populus trichocarpa (black cottonwood), or Oryza sativa (Asian rice), having amino acid sequences as set forth in FIGS. 15A-H and FIG. 16A-B, and a VAS1 enzyme having an amino acid sequence which is at least about 90 percent or at least about 95 percent homologous (as determined using software such as BLAST or FASTA) to the amino acid sequence of the VAS1 of any of the aforelisted plants.

In certain non-limiting embodiments, the mutation in the active site is a mutation of an amino acid selected from the group consisting of Met19, Lys233, Ile267 and R362 in a VAS1 enzyme having an amino acid sequence selected from the group of sequences having NCBI Accession number NCBI Accession No. NP_{—}178152 (Arabidopsis thaliana Arabidopsis thaliana), NCBI Accession No. XP_{—}002887833 (Arabidopsis lyrata subsp. lyrata), NCBI Accession No. XP_{—}002517536 (Ricinus communis), NCBI Accession No. XP_{—}002284955 (Vitis vinifera), NCBI Accession No. XP_{—}002284514 (Vitis vinifera), NCBI Accession No. XP_{—}002299622 (Populus trichocarpa), NCBI Accession No. NP_{—}001042188 (Oryza sativa) and a VAS1 enzyme having an amino acid sequence which is at least about 90 percent or at least about 95 percent homologous (as determined using software such as BLAST or FASTA) to any of the aforelisted amino acid sequences.

In certain non-limiting embodiments, the mutation in the active site is a mutation of Ile267 in a VAS1 enzyme from a plant selected from the group of Arabidopsis thaliana, Arabidopsis lyrata subsp. lyrata, Catharantus roseus (Madagasar rosy periwinkle), Solanum lycopersicum (tomato), Gossypium hirsutum (cotton), Zea mays (corn), Brachypodium distachyon (purple false brome), Selaginella moellendorfii, Ricinus communis (castor oil plant), Vitis vinifera (common grape vine), Populus trichocarpa (black cottonwood), Oryza sativa (Asian rice), for example having an amino acid sequence as set forth in FIGS. 15A-H and/or 16A-B, and a VAS1 enzyme having an amino acid sequence which is at least about 90 percent or at least about 95 percent homologous (as determined using software such as BLAST or FASTA) to the amino acid sequence of the VAS1 of any of the aforelisted plants.

In certain non-limiting embodiments, the mutation in the active site is a mutation of Ile267 in a VAS1 enzyme having an amino acid sequence selected from the group of sequences having NCBI Accession number NCBI Accession No. NP_{—}178152 (Arabidopsis thaliana Arabidopsis thaliana), NCBI Accession No. XP_{—}002887833 (Arabidopsis lyrata subsp. lyrata), NCBI Accession No. XP_{—}002517536 (Ricinus communis), NCBI Accession No. XP_{—}002284955 (Vitis vinifera), NCBI Accession No. XP_{—}002284514 (Vitis vinifera), NCBI Accession No. XP_{—}002299622 (Populus trichocarpa), NCBI Accession No. NP_{—}001042188 (Oryza sativa) and a VAS1 enzyme having an amino acid sequence which is at least about 90 percent or at least about 95 percent homologous (as determined using software such as BLAST or FASTA) to any of the aforelisted amino acid sequences.

In certain non-limiting embodiments, the mutation is Met19 to arginine, histidine, lysine, aspartic acid, glutamic acid, serine, threonine, asparagine, glutamine, alanine, valine, isoleucine, leucine, phenylalanine, tyrosine, or tryptophan.

In certain non-limiting embodiments, the mutation is Lys233 to arginine, histidine, aspartic acid, glutamic acid, serine, threonine, asparagine, glutamine, alanine, valine, isoleucine, leucine, methionine, phenylalanine, tyrosine, or tryptophan.

In certain non-limiting embodiments, the mutation is Ile267 to arginine, histidine, lysine, aspartic acid, glutamic acid, serine, threonine, asparagine, glutamine, alanine, valine, leucine, methionine, phenylalanine, tyrosine, or tryptophan. In certain non-limiting embodiments, the mutation is Ile267 to methionine. In certain non-limiting embodiments, the mutation is Ile267 to leucine.

In certain non-limiting embodiments, the mutation is Arg362 to histidine, lysine, aspartic acid, glutamic acid, serine, threonine, asparagine, glutamine, alanine, valine, isoleucine, leucine, methionine, phenylalanine, tyrosine, or tryptophan.

In certain non-limiting embodiments, the invention provides for an Arabidopsis thaliana plant having a VAS1 protein having a sequence as set forth in NCBI Accession No. NP_{—}178152, and containing a Ile267Met mutation.

In certain non-limiting embodiments, the invention provides for an Arabidopsis lyrata subsp. lyrata plant having a VAS1 protein having a sequence as set forth in NCBI Accession No. XP_{—}002887833, and containing a Ile267Met mutation.

In certain non-limiting embodiments, the invention provides for a Ricinus communis plant having a VAS1 protein having a sequence as set forth in NCBI Accession No. XP_{—}002517536, and containing a Ile267Met mutation.

In certain non-limiting embodiments, the invention provides for a Vitis vinifera plant having a VAS1 protein having a sequence as set forth in NCBI Accession No. XP_{—}002284955 or XP_{—}002284514, and containing a Ile267Met mutation.

In certain non-limiting embodiments, the invention provides for a Populus trichocarpa plant having a VAS1 protein having a sequence as set forth in NCBI Accession No. XP_{—}002299622, and containing a Ile267 Met mutation.

In certain non-limiting embodiments, the invention provides for a Oryza sativa plant having a VAS1 protein having a sequence as set forth in NCBI Accession No. NP_{—}001042188, and containing a Ile267Met mutation.

In certain non-limiting embodiments, the mutation in the active site is a mutation, to another naturally occurring amino acid, of an amino acid selected from the group consisting of Lys248, Ile313, R414 or a combination thereof in a VAS1 enzyme from Chlamydomonas reinhardtii, for example having a sequence as set forth in FIG. 15I, or a VAS1 enzyme having an amino acid sequence which is at least about 90 percent or at least about 95 percent homologous (as determined using software such as BLAST or FASTA) thereto.

In certain non-limiting embodiments, the invention provides for a nucleic acid encoding any of the above described VAS1 mutants. Said nucleic acid may be prepared using methods known in the art, for example using publicly available nucleic acid sequences encoding said enzymes or synthetic sequences and introducing mutations described herein. In certain non-limiting embodiments, said nucleic acid is operably linked to a promoter that is active in a host plant of interest, as are known in the art. In certain non-limiting embodiments, the promoter may be an inducible promoter. In certain non-limiting embodiments, the promoter may be an auxin-inducible promoter. Said nucleic acid may be introduced into a host plant to produce a transgenic plant, using methods known in the art.

### 5.3 Plants Carrying VAS1 Mutations

In certain non-limiting embodiments, the invention provides for a transgenic plant carrying, as a transgene, a nucleic acid encoding a VAS1 mutant as set forth above.

In certain, non-limiting embodiments, the plant is a crop plant, for example a plant selected from the group consisting of a rice plant, a corn plant, a wheat plant, a cotton plant, a castor oil plant, a tomato plant, a tobacco plant, an oat plant, a barley plant, a soybean plant, a grape plant, or a hemp plant.

In certain non-limiting embodiments the plant is a decorative plant.

In certain non-limiting embodiments the plant is a tree, such as an oak tree, a cherry tree, an apple tree, a poplar tree, a pear tree, a maple tree, an elm tree, a black cottonwood tree, a chestnut tree or a pine tree.

### 5.4 Methods of Modulating Plant Growth

In certain non-limiting embodiment, the present invention provides for methods of modulating plant growth, in particular in response to shade, for example shade from another plant, comprising introducing, into the plant, a mutant VAS1 gene as set forth above.

In certain non-limiting embodiments, the present invention provides a method for modulating plant growth, comprising introducing, into a plant of interest, a mutant VAS1 gene to produce a transgenic plant wherein, after the transgenic plant is exposed to shade, the amount of auxin in the transgenic plant is reduced by a factor X relative to a comparable non-transgenic plant but the amount of ethylene in the transgenic plant is increased, not reduced, or reduced by a factor at least 30% less than X relative to a comparable non-transgenic plant.

In certain non-limiting embodiments, the present invention provides a method for modulating plant growth, comprising introducing, into a plant of interest, a mutant VAS1 gene to produce a transgenic plant wherein, after the transgenic plant is exposed to shade, the amount of ethylene in the transgenic plant is reduced by a factor Y relative to a comparable non-transgenic plant but the amount of auxin in the transgenic plant is increased, not reduced, or reduced by a factor at least 30% less than Y relative to a comparable non-transgenic plant.

In certain non-limiting embodiments, the present invention provides a method for improving crop yield, comprising cultivating crop plants comprising a plurality of transgenic plants carrying a mutant VAS1 transgene that modulates the response of the transgenic plant to shade, as set forth herein, wherein when a harvest is obtained from said crop plants, the yield is improved relative to a harvest obtained from crop plants that do not comprise transgenic plants carrying the mutant VAS1 transgene.

### 6. EXAMPLE 1

### Structure-Guided Synthetic Aminotransferase Decouples Linked Biosynthetic Pathways of Hormones

6.1 Materials and Methods

Plant Growth Conditions, IAA and ACC Measurement.

The growth conditions for plants, and the approaches for quantifying the levels of IAA and ACC are same as described in reference 10.

Protein Expression, Purification and Mutagenesis.

The coding sequence of AtVAS1 was inserted between the NcoI and XhoI sites of the expression vector pHIS8 (“HIS8” disclosed as SEQ ID NO: 1) (a modified version of pET28a (+) containing an N-terminal 8-histidine tag (SEQ ID NO: 1)), which under the control of a T7 promoter. E. coli (BL21) cells harboring the AtVAS1 expression vector were grown in Terrific Broth at 37° C. until reaching an OD600 nm of 1.0 then cooled down to 18° C. and induced with 0.5 mM isopropyl-b-D-thiogalactoside (IPTG) and allowed to grow overnight at 18° C. for approximately 12-14 hr after. Cells were harvested by centrifugation and resuspended in lysis buffer (50 mM Tris-HCl, pH 8.0, 500 mM NaCl, 20 mM imidazole, 1% (v/v) Tween-20, 10% (v/v) glycerol, 10 mM 2-mercaptoethanol) containing lysozyme (0.5 mg/ml) at 4° C. for 1 hr and lysed by sonication. The lysate was centrifuged at 18,000 rpm for 45 min at 4° C. and then the supernatant were passed over Ni2+-NTA agarose (Qiagen). The AtVAS1 protein was eluted with lysate buffer supplemented with 250 mM imidazole after washed with wash buffer (lysis buffer without Tween-20 and glycerol). The N-terminal tag was cleaved by treatment with thrombin while dialyzing overnight in dialysis buffer (50 mM Tris-HCl, (pH8.0), 500 mM NaCl, 20 mM 2-mercaptoethanol). The thrombin and uncut AtVAS1 was removed by passing over Benzamidine Sepharose 4 Fast Flow (high sub) (GE Healthcare) and Ni2+-NTA agarose. AtVAS1 was further purified by size-exclusion chromatography using a Superdex 200 HR16/60 column. AtVAS1 fractions were combined, concentrated to approximately 12 mg ml-1 and frozen at −80° C.

AtVAS1 variants were made using the QuickChange protocol with PfuTurbo® DNA Polymerase (Stratagene) together with a 6.5 min PCR extension time on AtVAS1 pHIS8 (“HIS8” disclosed as SEQ ID NO: 1). The primer pairs used in all PCR reactions are listed in Table 2. The mutant proteins were expressed and purified as described for wild-type protein.

Crystallization and Data Collection.

Crystals of VAS1 were grown by hanging-drop vapor diffusion at 4° C. using 2 μl drop containing 1 μl of VAS1 (10-14 mg ml-1 in 12.5 mM Tris-HCl, pH 8.0, 50 mM NaCl, 2 mM dithiothrietol) and 1 μl of reservoir. VAS1 crystals formed large plates over a reservoir contained of 20% PEG 3,350, 3M cesium chloride and 0.1M sodium succinate at pH5.5. Crystals grew within 2d and were improved by streak seeding. For co-crystals of VAS1•PLP, VAS1•PLP•KMBA and VAS1•PLP•IAA, the protein solution included 0.5 mM PLP or 0.5 mM PLP plus 10 mM KMBA or 0.5 mM PLP plus 10 mM IAA.

Crystals were flash frozen by immersion in liquid nitrogen following 10 s incubations in a cryoprotectant solution consisting of reservoir solution supplemented with 17% (vol/vol) ethylene and 0.5M sodium ascorbate. X-ray data were collected at 110 K on ALS beamlines 8.2.1 and 8.2.2 (Lawrence Berkeley National Laboratory, Berkeley, Calif.) using an ADSC Q315 CCD detector. All x-ray diffraction data were collected at λ=1.0 Å.

X-Ray Structure Determination.

The observed reflections were indexed, integrated and scaled using iMosflm and SCALA in the CCP4 suite. The starting search model for MR was a homology model for a monomer of the AtVAS1 protein, based on the structure of aminotransferase TM1255 (PDB entry1O4S) and constructed with the Modeler program (Sali and Blundell, 1993). Initial models for VAS1 were obtained by rotational and translational searches using Phenix molecular replacement program. It was then refined with the simulated annealing, individual B factor and non crystallographic symmetry restraints refinement in Phenix. Coot was used for visualization of calculated electron density maps and manual rebuilding of atomic models. Programs from the CCP4 suite were employed for all other crystallographic calculations. Molecular graphics were generated with the program PyMol.

Enzyme Activities Assays.

VAS1 and its variants transaminase activity was monitor by HPLC-MS with an Agilent 1100 series LC-MSD instrument and an Agilent Zorbax Eclipse XDB-C18 (4.6×150 mm, 5-mm particle size) reversed-phase column. Chromatographic separations employed a flow rate of 0.5 mL min-1 and a linear gradient with initial and final mobile phases consisting of 95% water: 4.9%: acetonitrile: 0.1% formic acid (v/v/v), and 5% water: 94.9% acetonitrile: 0.1% formic acid (v/v/v), respectively. VAS1 mutants relative activity assays were conducted in a 200 μL volume containing 50 mM L-Met (with the exception of L-Trp), 10 mM IPA, 200 μM PLP and 10 μg of purified VAS1 or VAS1 variants protein in a reaction buffer consisting of 50 mM K₂HPO₄/KH₂PO₄ (pH 8.5). To further test the best amino donor of I267M and I267F mutant, one of 19 other amino acids (with the exception of L-Trp) was taking the place of L-Met. To examine I267M variant's best amino acceptor, we fixed His and provide 9 different α-ketoacid (glyoxylate, pyruvate, 2-ketobutyrate, KMBA, 2-oxoglutarate, oxaloacetate, phenylpyruvate, 4-hydroxyphenylpyruvate and IPA), PLP and protein are same amount with the typical assays. Reactions were kept at 30° C. for 1 h and were terminated by rapid removal of VAS1 using ultrafiltration through Amicon filters at 4° C. (10 kD cutoff membrane). 10 μl Amicon filtered samples were injected in the HPLC-MS. Relative specific activities were calculated after integration of the Trp peak (based on the absorbance at 254 nm) and the activity against Met and IPA was arbitrarily set to 100% for VAS1 variants M19V, M19I, M19F, I267V, 1267 M and I267F. For I267M mutant relative activity, the activity against His and IPA was arbitrarily set to 100%.

For VAS1 I267M steady-state kinetic assays, 1 mL assay mixture contained 100 mM sodium phosphate buffer (pH 8.0), 0.4 μM recombinant VAS1 I 267M, 100 μM PLP and variable amounts of His (0.1 to 10 mM) and IPA (10 to 200 μM). The reactions were incubated at 23° C. and the OD321 nm were measured at 2-min, 5-min, 8-min, 10-min, 12-min and 15-min time points to monitor the linear rate of loss of IPA. Absorbance values were corrected to changes in IPA concentration based on an IPA standard curve. At 321 nm, absorbance from Trp was negligible. K_{M}, k_{cat} and Vₘₐₓ were calculated by nonlinear regression analysis using GraphPad Prism 5 fits to the Michaelis-Menten equation.

6.2 Results and Discussion

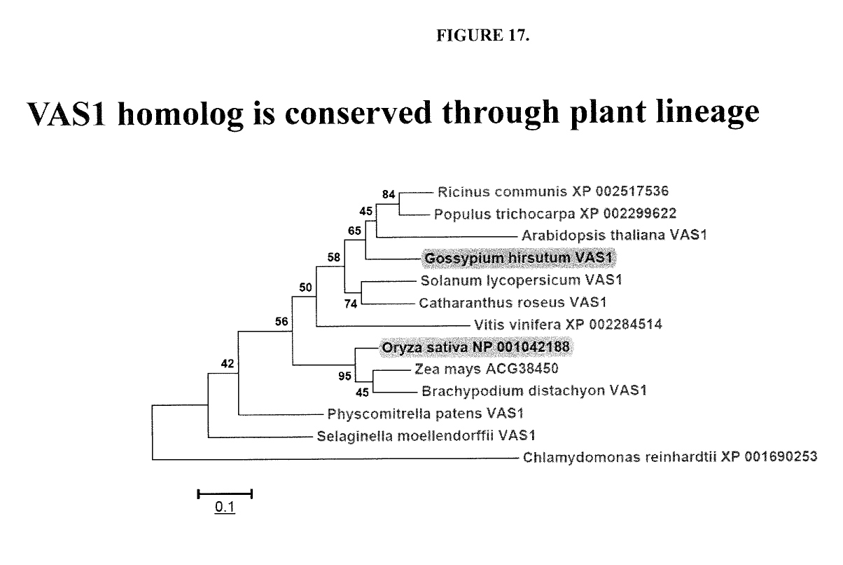

The VAS1 homolog is conserved through plant lineages (FIG. 17). To precisely design VAS1 variants with altered substrates specificity, we explored the details of the substrates binding pocket of VAS1 by solving the X-ray crystal structures of VAS1 with cofactor PLP as well as the complexes containing VAS1, PLP and substrates or products (Table 1).

The atomic resolution structure of VAS1•PLP complex showed that VAS1 forms a symmetric dimer, with each monomer containing an active-site pocket around the 2-fold axis (FIG. 4). The active-site pocket located at the interface of small domain (resi7-65, 270-392, α1-α4, α13-α18,β8-β9) and large domain (resi 66-269, α5-α12,β1-β7) of one monomer (FIG. 4). Helixes α1-4, α6-7 and α12-13 correspond to the dimer interface interaction and the interface area is about 3065 Å2 (FIG. 4). The core of large domain contains the sharply twisted seven-stranded β sheets (the sheets are parallel except β7 arranged as β1↑-β7↓-β6↑-β5↑-β4↑-β2↑-β3↑) that are surrounded by 8 α helixes. The 8 α helixes and 7 β sheets make αβα sandwich architecture (FIG. 4). The cofactor PLP is located at the bottom of the active-site cavity with its si-face directed toward the protein side and serve as an electron sink (FIGS. 4 and 5). PLP forms an internal aldimine bond with the ε-amino group of Lys 233 in the large domain and interacts extensively with the residues located at or near one end of the seven-stranded sheet of the large domain. Specifically, Asn 176 and Tyr 207 interact with the phenoic oxygen O3 of PLP with distance of 2.97 A (O3′-NH2) and 2.91 A (03′-OH), respectively (FIG. 4). Asp 204 interacts with the pyridine nitrogen of the cofactor PLP, thus maintaining the cofactor in the protonated form (FIG. 4)¹⁸. The negative charge of the phosphate group of PLP is well balanced with the positive charge of Arg 241 (FIG. 4). Pyridine ring of PLP is sandwiched by the 4-hydroxyphenyl ring of Tyr 123 and hydroxy side-chain of Thr 206 (FIG. 4). In general, the VAS1•PLP structure revealed that VAS1 possesses similar tertiary and quaternary structures as several other aminotransferases like pig cytosolic¹⁹ and chicken mitochondrial aspartate transaminase (AspATs)²⁰. Thus, the structure of VAS1•PLP doesn't provide sufficient information about how VAS1 recognizes the specific substrates. Therefore, we moved on to investigate the structures of VAS1 complexes harboring substrates/products.

Unlike other characterized aminotransferases that show low substrates specificityl^{4,18}, VAS1 has relative high selectivity for substrates, with Met as the best amino donor and 3-IPA the best amino acceptor¹⁰. So, it would be interesting to dissect how two structurally distinct substrates, Met with a thioether chain and 3-IPA with a indole ring, are recognized by the VAS1's active site, while the other amino acids and α-keto acids are more or less discriminated against by VAS1. We failed to co-crystallize VAS1 with the substrate Met likely due to that VAS1 is a highly efficient enzyme, but obtained the VAS1 complex with the cofactor PLP and the product KMBA (VAS1•PLP•KMBA). The other VAS1 substrate 3-IPA is redox-sensitive and unstable^{21,22}, so we turned to its analog IAA and succeeded in co-crystallizing VAS1 with IAA and the cofactor PLP (VAS1•PLP•IAA). We were able to examine in detail how VAS1 binds to the substrates Met and 3-IPA based on the structures of VAS1•PLP•KMBA complex and VAS1•PLP•IAA complex, respectively, since the existence of thioether chain in both Met and KMBA, and indole ring in both 3-IPA and IAA. The atomic resolution structures revealed that in these complexes, cofactor PLP binds to VAS1 in the same way as that in VAS1•PLP structure. Interestingly, comparison of VAS1•PLP•KMBA to VAS1•PLP•IAA complex showed that, although structurally quite distinct, KMBA and IAA bind to VAS1 in almost the same way (FIG. 2A): both are recognized by Met 19, Ile 267* (asterisk indicates the residue from another subunit of the dimmer unit) and Arg 362 (FIGS. 1B, 1C, 2A, 6 and 7); the conserved Arg 362 guanidine group tethers the carboxylate group of both KMBA and IAA by making salt bridge with an “end-on” geometry (FIGS. 1B, 1C, 2A); both the thioether moiety of KMBA and the indole ring of IAA formed non polar interactions with the side chains of Met 19 and Ile 267*(FIGS. 1B, 1C, 2A, 6 and 7) (asterisk indicates the residue from another subunit of the dimer). These results suggest that aminotransferase VAS1 recognized two substrates Met and 3-IPA by the same interaction network in the same binding pocket. Such mechanism is quite unique and distinct from the mechanisms used by all the crystallographically characterized aminotransferases to recognize their paired substrates¹⁸. For example, the histidinol-phosphate aminotransferase recognizes the acidic side chain of glutamate and the basic side chain of histidinol phosphate at different positions of the active site by inducing flexible loop's movement²³; and the aromatic-amino-acid aminotransferases rely on rearrangement of the hydrogen bond network of the active site without a conformational change in the backbone structure of the enzymes²⁴.

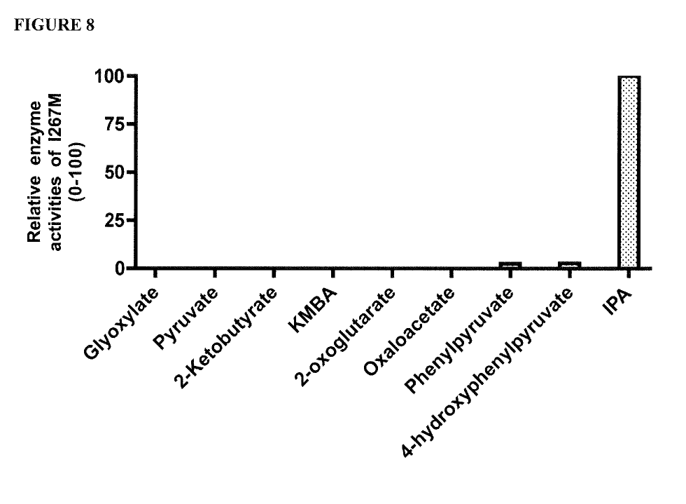

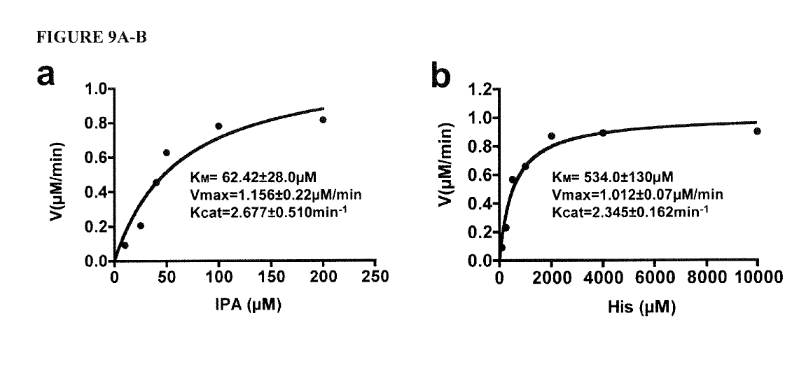

The three-dimensional structures reveal Met 19 and Ile 267 have strong non-polar interactions with both the thioether moiety of KMBA and the indole ring of IAA (FIGS. 1B, 1C, 2A). Thus, these two residues serve as the ideal targets to probe potential VAS1 variants that may be capable of using other amino acids rather than Met as the efficient amino donor. Therefore, we generated Met19Val, Met19Ile, Met19Phe, Ile267Val, Ile267Phe and Ile267Met variants to either expand or shrink the size of the VAS1's substrates binding pocket. Notably, all the six VAS1 mutants expressed very well in E. coli cells and were purified to homogeneity. Then, we performed enzyme activity assays of the invented VAS1 mutants as well as VAS1 by using fixed amount of Met as amino donor, 3-IPA as amino acceptor and PLP co-factor. The results showed that the relative activities of engineered VAS1 mutants including Met19Val, Met19Ile, Met19Phe, Ile267Val, Ile267Phe (hereafter, I267F) and Ile267Met (hereafter, I267M), are 69.1%, 77.6%, 45.8%, 74.8%, 2.8% and 2.1%, to those of VAS1 (FIG. 2B), respectively. Of our great interest are the I267F and I267M mutants that only maintained marginal VAS1 activities against Met. However, I267F mutant didn't show any significant aminotransferase activities using 19 different natural amino acids (except for Trp) as amino donor and 3-IPA as amino acceptor, indicating I267F mutation likely abolished the VAS1 enzyme activities. Interestingly, further in vitro functional analysis of VAS1 I267M variant demonstrated that unlike VAS1 preferring Met as the most efficient amino donor (FIG. 2C)¹⁰, VAS1 I267M choose Histidine (His) as the favorite amino donor (FIG. 2D). More importantly, VAS1 I267M retained 3-IPA as the best amino acceptor (FIG. 8). Furthermore, under steady-state conditions in vitro, I267M exhibited an apparent K_{M} for 3-IPA of 62.4 μM and an apparent K_{M} for His of 534 μM (FIG. 9A-B), which is comparable to VAS1's K_{M} for 3-IPA and Met¹⁰. The corresponding apparent Kcat values were 2.68 min⁻¹ and 2.35 min⁻¹ for 3-IPA and His, respectively (FIG. 9A-B).

These results suggest that the directly evolved VAS1 I267M, with altered substrate binding pocket size, is still a functional and efficient enzyme with 3-IPA (the auxin biosynthetic intermediate) as the best amino acceptor, and His rather than Met (the ethylene biosynthetic intermediate) as the best amino donor (FIGS. 2E and 2F). Thus, in vitro, VAS1 I267M could still convert 3-IPA to Trp linked to for auxin metabolism, but lost the ability to convert Met to KMBA linked to ethylene metabolism, thus successfully decoupling the VAS1's dual roles in metabolically regulating the homeostasis of both auxin and ethylene (FIGS. 2E and F).

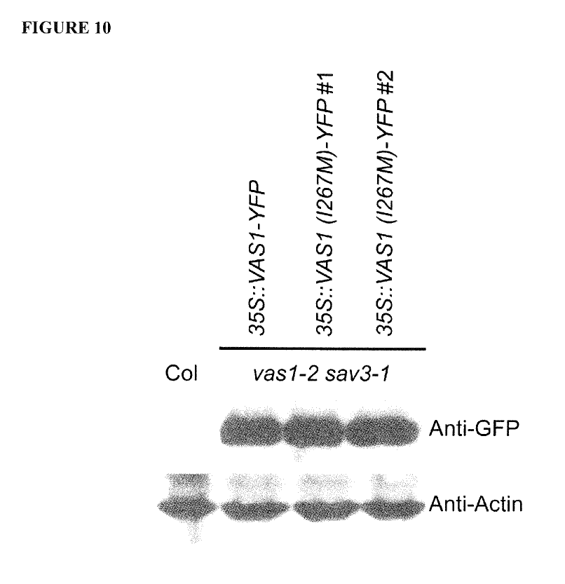

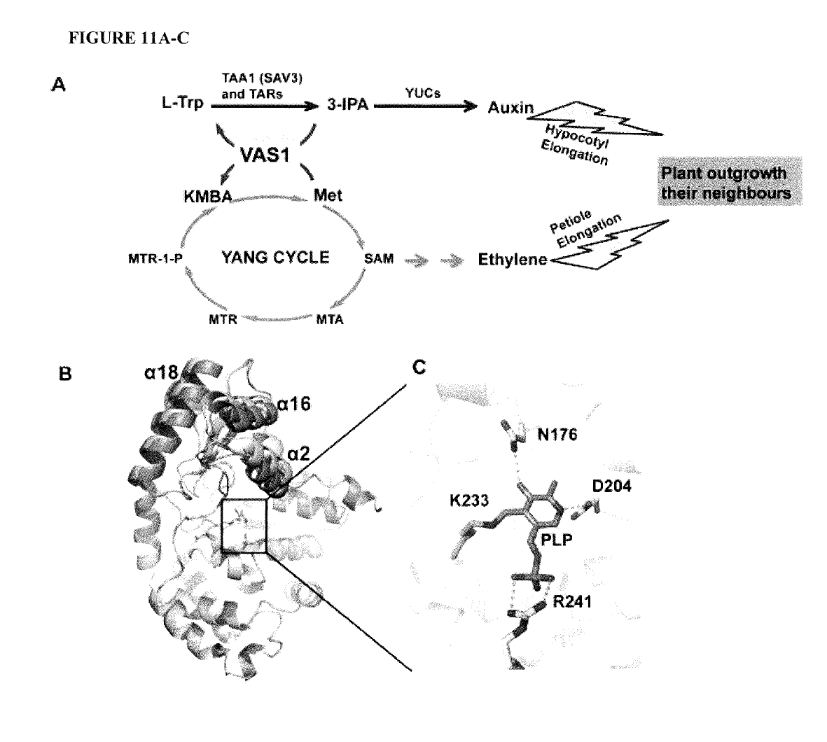

Next, we sought to investigate the directly evolved VAS1 I267M's biochemical and biological function in vivo by transforming 35S::VAS1(I267M)-GFP into vas1-2 sav3-1 double mutant background. Two independent resultant transgenic lines that accumulated the amount of VAS1(I267M) protein similar to the amount of VAS1 protein accumulated in the 35S:: VAS1-GFP transgenic lines (also in the vas1-2 sav3-1 double mutant background) were chosen for detailed phenotypic analyses (FIG. 10). Compared with sav3-1 single mutant, the vas1-2 sav3-1 double mutant possesses higher amount of both auxin and 1-aminocyclopropane-1-carboxylate (ACC, the stable penultimate precursor of ethylene) that are responsible for the double mutant's longer hypocotyls and petioles, respectively¹⁰. As reported before¹⁰, the 35S::VAS1-GFP transgene suppressed the longer hypocotyls and petioles of the vas1-2 sav3-1 double mutant (FIGS. 3A-C). Consistently, the 35S::VAS1-GFP transgene also brought down the higher levels of both auxin and ethylene in the vas1-2 sav3-1 double mutant to the levels similar to those in sav3-1 single mutant (FIGS. 3A, 3C and 3E)¹⁰. These results indicate that the VAS1-GFP transgene appropriately coordinates the metabolisms of auxin and ethylene (FIGS. 1A and 2E). By contrast, although the 35S::VAS1 (I267M)-YFP transgene could still repress the longer hypocotyls and higher levels of auxin of the vas1-2 sav3-1 double mutant indicating that VAS1 I267M is still be able to convert 3-IPA to Trp to regulate the auxin metabolisms in vivo (FIGS. 3A, 3B and 3D), the 35S::VAS1 (I267M)-YFP transgene didn't affect the longer petioles and higher amount of ethylene of vast-2 sav3-1 double mutant (FIGS. 3A, 3C and 3E) demonstrating that VAS1 I267M couldn't modulate the ethylene metabolisms in vivo likely due to its inability to efficiently convert Met into KMBA (FIGS. 2b, 2d and 2f). These in planta results are consistent with the enzymatic assays performed in vitro—both argue that VAS1 I267M, unlike VAS1 itself that can concurrently regulate the metabolisms of both auxin and ethylene (FIGS. 1A and 1E), could only modulate the auxin metabolism by retaining the 3-IPA as the amino acceptor, but lost the power to modulate the ethylene metabolism for being deprived of the ability to efficiently use Met as the amino donor (FIG. 2F).

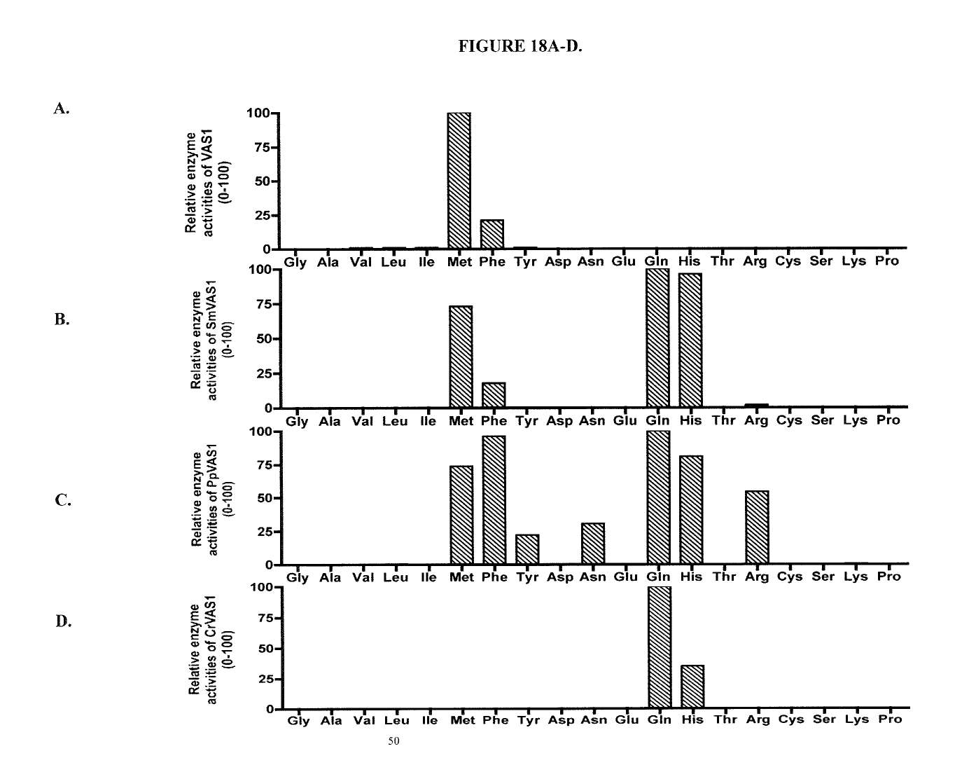

In summary, atomic resolution of enzyme-cofactor-substrates (products) complexes allows us to elegantly engineer aminotransferase VAS1, evolving a novel VAS1 I267M protein that successfully uncoupled the dual roles of VAS1 in coordinating the biosynthesis of auxin and ethylene without losing protein stability both in vitro and in vivo (FIG. 2f). This structure-based, metabolic engineering-aimed study promises the possibility to flexibly manipulate the levels of phytohormones auxin and ethylene for improving the fitness and adaptive ability of plants^{1,2,4}. Lower plants such as Selaginella moellendorfii, Phsycomitrella patens and Chlamydomonas reinhardtii demonstrate natural variation that decouples IA and Met recognition (FIG. 18A-D).

TABLE 1
Data collection and refinement statistics (molecular replacement)

| | | |
|---|---|---|
| | | VAS1-PLP- |
| VAS1-PLP | VAS1-PLP-IAA | KMBA |

Data collection

| | | | |
|---|---|---|---|
| Space group | P2₁ | P2₁ | P2₁2₁2₁ |
| Cell dimensions | | | |
| a, b, c (Å) | 68.59, 173.52, | 68.69, 173.91, | 77.19, 119.02, |
| | 72.97 | 73.30 | 171.58 |
| a, b, g (°) | 90, 113.6, 90 | 90, 114.26, 90 | 90, 90, 90 |
| Resolution (Å) | 57.84-1.91 | 59.49-2.05 | 51.71-1.86 |
| | (2.02-1.91) | (2.16-2.05) | (1.96-1.86) |
| R_{merge} | 0.097(0.501) | 0.103(0.344) | 0.134(0.40) |
| I/sI | 8.3(1.4) | 7.0/1.4 | 12.6(2.0) |
| Completeness (%) | 82.5(69) | 77.3/55.4 | 92.8(62.2) |
| Redundancy | 3.1(1.8) | 2.4/1.1 | 10.5(2.8) |

Refinement

| | | | |
|---|---|---|---|
| Resolution (Å) | 52.96-1.91 | 37.57-2.05 | 51.69-1.86 |
| No. reflections | 98611 | 75696 | 123386 |
| R_{work}/R_{free} | 0.181/0.225 | 0.177/0.227 | 0.161/0.207 |
| No. atoms | | | |
| Protein | 12165 | 12148 | 12202 |
| Ligand/ion | 60 | 112 | 96 |
| Water | 1160 | 1020 | 1864 |
| B-factors | | | |
| Protein | 22.3 | 24.3 | 15.1 |
| Ligand/ion | 18.3 | 24.8 | 14.5 |
| Water | 29.2 | 29.3 | 26.7 |
| R.m.s. deviations | | | |
| Bond lengths (Å) | 0.010 | 0.009 | 0.009 |
| Bond angles (°) | 1.143 | 1.145 | 1.127 |

TABLE 2
Primers for the VAS1 variants

| | |
|---|---|
| M19I | 5′-CTGATATGCCCGTCATCGCTCAGATTCGGAG |
| | (SEQ ID NO: 2) |
| | 5′-CTCCGAATCTGAGCGATGACGGGCATATCAG |
| | (SEQ ID NO: 3) |
| M19V | 5′-CACTGATATGCCCGTCGTGGCTCAGATTCGGAG |
| | (SEQ ID NO: 4) |
| | 5′-CTCCGAATCTGAGCCACGACGGGCATATCAGTG |
| | (SEQ ID NO: 5) |
| M19F | 5′-GCACTGATATGCCCGTCTTCGCTCAGATTCGGAGTTT |
| | (SEQ ID NO: 6) |
| | 5′-AAACTCCGAATCTGAGCGAAGACGGGCATATCAGTGC |
| | (SEQ ID NO: 7) |
| I267V | 5′-TGAAAATTCAGGACAACATCCCAGTCTGTGCTGCCAT |
| | (SEQ ID NO: 8) |
| | 5′-ATGGCAGCACAGACTGGGATGTTGTCCTGAATTTTCA |
| | (SEQ ID NO: 9) |
| I267F | 5′-TGAAAATTCAGGACAACATCCCATTCTGTGCTGCCAT |
| | (SEQ ID NO: 10) |
| | 5′-ATGGCAGCACAGAATGGGATGTTGTCCTGAATTTTCA |
| | (SEQ ID NO: 11) |
| I267M | 5′-TGAAAATTCAGGACAACATCCCAATGTGTGCTGCCATAA |
| | (SEQ ID NO: 12) |
| | 5′-TTATGGCAGCACACATTGGGATGTTGTCCTGAATTTTCA |
| | (SEQ ID NO: 13) |

### 7. EXAMPLE 2

### Decoupling the Auxin and Ethylene Metabolic Link Via Structure-based Engineering of the Aminotransferase VAS1

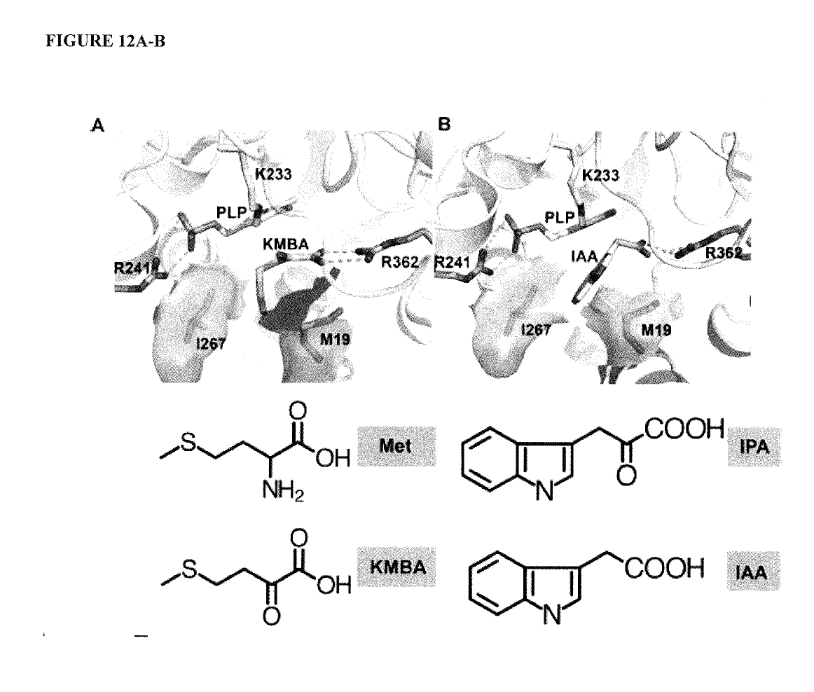

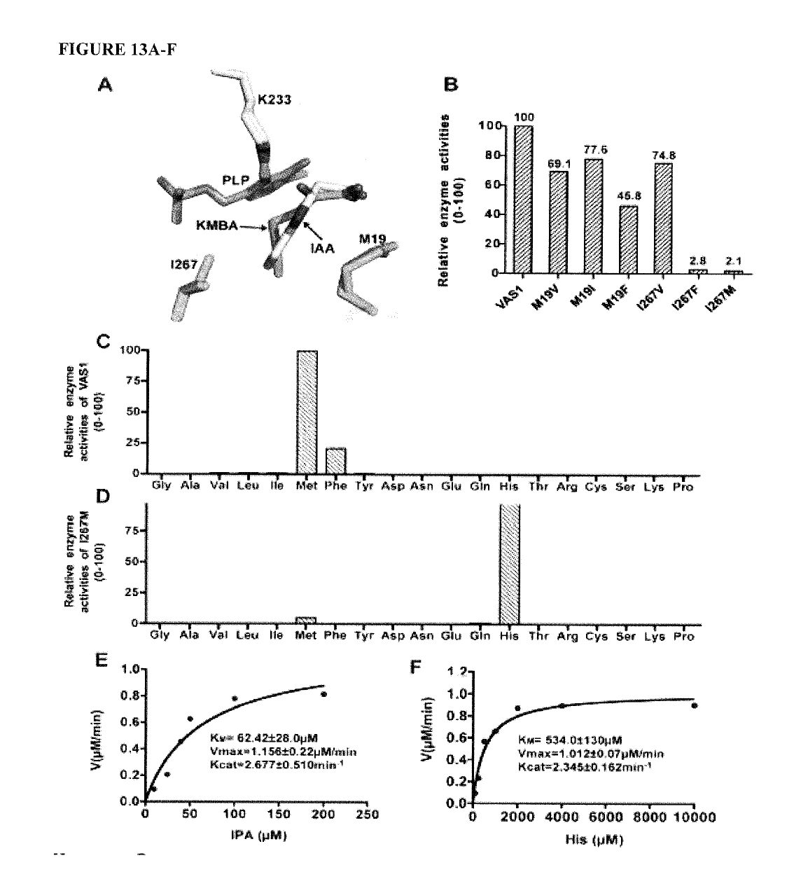

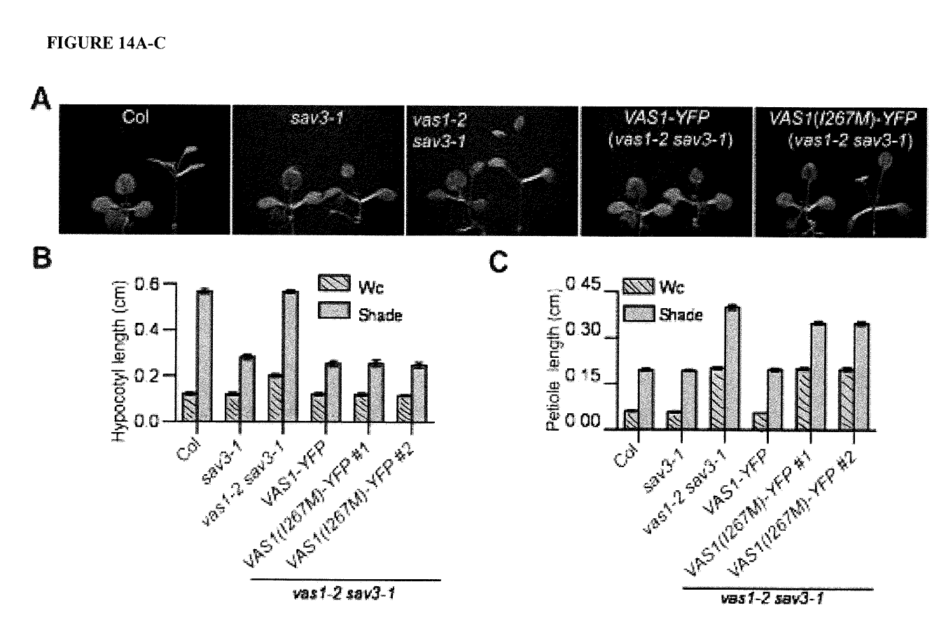

Auxin and ethylene are two fundamental phytohormones that play vital roles in various plant growth and developmental processes. VAS1, a pyridoxalphosphate-dependent aminotransferase, metabolically links auxin andethylene biosynthesis by using L-Met (ethylene biosynthetic intermediate) as an amino donor and indole-3-pyruvic acid (3-IPA) as an amino acceptor to produce L-Trp and KMBA (FIG. 11A). Here we report the crystal structures of VAS1 (FIGS. 11B and C) and the structure-based engineering of VAS1 to decouple its roles in auxin and ethylene biosynthesis. The structures of VAS1•PLP•IAA complex and VAS1•PLP•KMBA complex uncover that VAS1's two substrates 3-IPA and LMet share the same substrate binding pocket (FIGS. 12A and 12B). Based on its three dimensional structure, we successfully engineered VAS1 and showed that its I267M mutant decoupled the dual roles of VAS1 in coordinating auxin and ethylene biosynthesis both in vitro and in vivo (FIGS. 13A-F and 14A-C). Our findings prove that metabolic engineering strategies are powerful in manipulating the activities of auxin and ethylene and their crosstalk.

### 8. REFERENCES

The following are references referred to by superscript elsewhere in this document.
1. Santner, A., Calderon-Villalobos, L. I. & Estelle, M. Plant hormones are versatile chemical regulators of plant growth. Nat Chem Biol 5, 301-307, doi:10.1038/nchembio.165 nchembio.165 [pii] (2009).
2. Wolters, H. & Jurgens, G. Survival of the flexible: hormonal growth control and adaptation in plant development. Nat Rev Genet 10, 305-317, doi:10.1038/nrg2558 nrg2558 [pii] (2009).
3. Norman, A. W. & Litwack, G. Hormones. 2nd edn, (Academic Press, 1997).
4. Jaillais, Y. & Chory, J. Unraveling the paradoxes of plant hormone signaling integration. Nat Struct Mol Biol 17, 642-645, doi:10.1038/nsmb0610-642 nsmb0610-642 [pii] (2010).
5. Lee, J. W., Kim, T. Y., Jang, Y. S., Choi, S. & Lee, S. Y. Systems metabolic engineering for chemicals and materials. Trends Biotechnol 29, 370-378, doi:10.1016/j.tibtech.2011.04.001 S0167-7799(11)00065-5 [pii] (2011).
6. Shachar-Hill, Y. Metabolic network flux analysis for engineering plant systems. Curr Opin Biotechnol 24, 247-255, doi:10.1016/j.copbio.2013.01.004 S0958-1669(13)00007-4 [pii] (2013).
7. Nielsen, J. & Keasling, J. D. Synergies between synthetic biology and metabolic engineering. Nat Biotechnol 29, 693-695, doi:10.1038/nbt.1937 nbt.1937 [pii] (2011).
8. Pleiss, J. Protein design in metabolic engineering and synthetic biology. Curr Opin Biotechnol 22, 611-617, doi:10.1016/j.copbio.2011.03.004 S0958-1669(11)00043-7 [pii] (2011).
9. Boyle, P. M. & Silver, P. A. Parts plus pipes: synthetic biology approaches to metabolic engineering. Metab Eng 14, 223-232, doi:10.1016/j.ymben.2011.10.003 S1096-7176(11)00104-2 [pii] (2012).
10. Zheng, Z. et al. Coordination of auxin and ethylene biosynthesis by the aminotransferase VAS1. Nat Chem Biol 9, 244-246, doi:10.1038/nchembio.1178 nchembio.1178 [pii] (2013).
11. Ioannou, G. N. Implications of elevated serum alanine aminotransferase levels: think outside the liver. Gastroenterology 135, 1851-1854, doi:10.1053/j.gastro.2008.11.005 S0016-5085(08)01985-9 [pii] (2008).
12. Goessling, W. et al. Aminotransferase levels and 20-year risk of metabolic syndrome, diabetes, and cardiovascular disease. Gastroenterology 135, 1935-1944, 1944 e1931, doi:10.1053/j.gastro.2008.09.018 S0016-5085(08)01683-1 [pii] (2008).
13. Kim, H. C. et al. Normal serum aminotransferase concentration and risk of mortality from liver diseases: prospective cohort study. BMJ 328, 983, doi:10.1136/bmj.38050.593634.63 bmj.38050.593634.63 [pii] (2004).
14. Liepman, A. H. & Olsen, L. I. Genomic analysis of aminotransferases in Arabidopsis thaliana. Crit Rev Plant Sci 23, 73-89, doi:Doi 10.1080/07352680490273419 (2004).
15. Schindhelm, R. K. et al. Alanine aminotransferase predicts coronary heart disease events: a 10-year follow-up of the Hoorn Study. Atherosclerosis 191, 391-396, doi:S0021-9150(06)00194-8 [pii] 10.1016/j.atherosclerosis.2006.04.006 (2007).
16. Franklin, K. A. Shade avoidance. New Phytol 179, 930-944, doi:NPH2507 [pii] 10.1111/j.1469-8137.2008.02507.x (2008).
17. Casal, J. J. Shade avoidance. Arabidopsis Book 10, e0157, doi:10.1199/tab.0157 (2012).
18. Eliot, A. C. & Kirsch, J. F. Pyridoxal phosphate enzymes: mechanistic, structural, and evolutionary considerations. Annu Rev Biochem 73, 383-415, doi:10.1146/annurev.biochem.73.011303.074021 (2004).
19. Rhee, S. et al. Refinement and comparisons of the crystal structures of pig cytosolic aspartate aminotransferase and its complex with 2-methylaspartate. J Biol Chem 272, 17293-17302 (1997).
20. McPhalen, C. A., Vincent, M. G. & Jansonius, J. N. X-ray structure refinement and comparison of three forms of mitochondrial aspartate aminotransferase. J Mol Biol 225, 495-517, doi:0022-2836(92)90935-D [pii] (1992).
21. Poeggeler, B. et al. Indole-3-propionate: a potent hydroxyl radical scavenger in rat brain. Brain Res 815, 382-388, doi:S0006-8993(98)01027-0 [pii] (1999).
22. Chowdhury, G. et al. Structural identification of Diindole agonists of the aryl hydrocarbon receptor derived from degradation of indole-3-pyruvic acid. Chem Res Toxicol 22, 1905-1912, doi:10.1021/tx9000418 (2009).
23. Haruyama, K. et al. Structures of Escherichia coli histidinol-phosphate aminotransferase and its complexes with histidinol-phosphate and N-(5′-phosphopyridoxyl)-L-glutamate: double substrate recognition of the enzyme. Biochemistry 40, 4633-4644, doi:bi002769u [pii] (2001).
24. Okamoto, A., Nakai, Y., Hayashi, H., Hirotsu, K. & Kagamiyama, H. Crystal structures of Paracoccus denitrificans aromatic amino acid aminotransferase: a substrate recognition site constructed by rearrangement of hydrogen bond network. J Mol Biol 280, 443-461, doi:S0022-2836(98)91869-1 [pii] 10.1006/jmbi.1998.1869 (1998).

Various publications are cited herein, the contents of which are hereby incorporated by reference in their entireties. Various amino acid sequence and/or nucleic acid sequence database Accession Numbers are provided herein, the referenced sequences of which are incorporated herein by reference.

What is claimed is:

1. A mutant VAS1 enzyme having a mutation in the VAS1 active site that results in a modulation of the activity of VAS1, relative to unmutated enzyme, to convert Methionine to KMBA and/or to convert 3-IPA to L-Trp.

2. The mutant VAS1 enzyme of claim 1, wherein the mutation in the VAS1 active site reduces the activity of VAS1, relative to unmutated enzyme, to convert Methionine to KMBA and/or to convert 3-IPA to L-Trp.

3. A mutant VAS1 enzyme having a mutation in the VAS1 active site that, when the mutant VAS1 is expressed in a plant, results in a modulated response of said plant to shade.

4. The mutant VAS1 enzyme of claim 3, wherein expression of the mutant VAS1 in a plant results in a modulated response of said plant to shade wherein negative regulation of auxin or ethylene by the mutant VAS1 is altered.

5. The mutant VAS1 enzyme of claim 1, wherein the mutation in the active site is a mutation of an amino acid selected from the group consisting of Met19, Lys233, Ile267 and R362.

6. The mutant VAS1 enzyme of claim 5 wherein the mutation in the active site is a mutation of an amino acid selected from the group consisting of Met19, Lys233, Ile267 and R362 in a VAS1 enzyme from a plant selected from the group of Arabidopsis thaliana, Arabidopsis lyrata subsp. lyrata, Catharanthus roseus, Solanum lycopersicum, Gossypium hirsutum, Zea mays, Brachypodium distachyon, Selaginella moellendorfii, Ricinus communis, Vitis vinifera, Populus trichocarpa, Oryza sativa, and a VAS1 enzyme having an amino acid sequence which is at least about 90 percent or at least about 95 percent homologous to the amino acid sequence of the VAS1 of any of the aforelisted plants.

7. The mutant VAS1 enzyme of claim 3, wherein the mutation in the active site is a mutation of an amino acid selected from the group consisting of Met19, Lys233, Ile267 and R362.

8. The mutant VAS1 enzyme of claim 7, wherein the mutation in the active site is a mutation of an amino acid selected from the group consisting of Met19, Lys233, Ile267 and R362 in a VAS1 enzyme from a plant selected from the group of Arabidopsis thaliana, Arabidopsis lyrata subsp. lyrata, Catharanthus roseus, Solanum lycopersicum, Gossypium hirsutum, Zea mays, Brachypodium distachyon, Selaginella moellendorfii, Ricinus communis, Vitis vinifera, Populus trichocarpa, Oryza saliva, and a VAS1 enzyme having an amino acid sequence which is at least about 90 percent or at least about 95 percent homologous to the amino acid sequence of the VAS1 of any of the aforelisted plants.

9. A transgenic plant expressing a mutant VAS1 enzyme according to claim 1.

10. A method of modulating plant growth, comprising introducing, into the plant, a mutant VAS1 gene encoding a mutant VAS1 enzyme having a mutation in the VAS1 active site that results in a modulation of the activity of VAS1, relative to unmutated enzyme, to convert Methionine to KMBA and/or to convert 3-IPA to L-Trp.

11. The method of claim 10, wherein plant growth in response to shade is modulated.

12. The method of claim 11, wherein the shade is shade from another plant.

13. A method for improving crop yield, comprising cultivating crop plants comprising a plurality of transgenic plants carrying a mutant VAS1 transgene that modulates the response of the transgenic plants to shade, wherein a harvest obtained from said crop plants comprises a yield that is improved relative to a harvest obtained from crop plants that do not comprise transgenic plants carrying the mutant VAS1 transgene.


SEQUENCE LISTING (72 sequences)

SEQ ID NO: 1 - protein, 8 aa, Artificial Sequence
  Description of Artificial Sequence Synthetic 8xHis tag
HHHHHHHH

SEQ ID NO: 2 - DNA, 31 bp, Artificial Sequence
  Description of Artificial Sequence Synthetic primer
ctgatatgcccgtcatcgctcagattcggag

SEQ ID NO: 3 - DNA, 31 bp, Artificial Sequence
  Description of Artificial Sequence Synthetic primer
ctccgaatctgagcgatgacgggcatatcag

SEQ ID NO: 4 - DNA, 33 bp, Artificial Sequence
  Description of Artificial Sequence Synthetic primer
cactgatatgcccgtcgtggctcagattcggag

SEQ ID NO: 5 - DNA, 33 bp, Artificial Sequence
  Description of Artificial Sequence Synthetic primer
ctccgaatctgagccacgacgggcatatcagtg

SEQ ID NO: 6 - DNA, 37 bp, Artificial Sequence
  Description of Artificial Sequence Synthetic primer
gcactgatatgcccgtcttcgctcagattcggagttt

SEQ ID NO: 7 - DNA, 37 bp, Artificial Sequence
  Description of Artificial Sequence Synthetic primer
aaactccgaatctgagcgaagacgggcatatcagtgc

SEQ ID NO: 8 - DNA, 37 bp, Artificial Sequence
  Description of Artificial Sequence Synthetic primer
tgaaaattcaggacaacatcccagtctgtgctgccat

SEQ ID NO: 9 - DNA, 37 bp, Artificial Sequence
  Description of Artificial Sequence Synthetic primer
atggcagcacagactgggatgttgtcctgaattttca

SEQ ID NO: 10 - DNA, 37 bp, Artificial Sequence
  Description of Artificial Sequence Synthetic primer
tgaaaattcaggacaacatcccattctgtgctgccat

SEQ ID NO: 11 - DNA, 37 bp, Artificial Sequence
  Description of Artificial Sequence Synthetic primer
atggcagcacagaatgggatgttgtcctgaattttca

SEQ ID NO: 12 - DNA, 39 bp, Artificial Sequence
  Description of Artificial Sequence Synthetic primer
tgaaaattcaggacaacatcccaatgtgtgctgccataa

SEQ ID NO: 13 - DNA, 39 bp, Artificial Sequence
  Description of Artificial Sequence Synthetic primer
ttatggcagcacacattgggatgttgtcctgaattttca

SEQ ID NO: 14 - protein, 394 aa, Arabidopsis thaliana
MGSFGMLSRRTLGTDMPVMAQIRSLMAELTNPMSLAQGVVHWQPPQKALEKVKELVWDPIISSYGPDEGLPELRQALLKKLREENKLTNSQVMVTAGANQAFVNLVITLCDAGDSVVMFEPYYFNSYMAFQMTGVTNIIVGPGQSDTLYPDADWLERTLSESKPTPKVVTVVNPGNPSGTYVPEPLLKRIAQICKDAGCWLIVDNTYEYFMYDGLKHCCVEGDHIVNVFSFSKTYGMMGWRLGYIAYSERLDGFATELVKIQDNIPICAAIISQRLAVYALEEGSGWITERVKSLVKNRDIVKEALEPLGKENVKGGEGAIYLWAKLPEGHRDDFKVVRWLAHRHGVVVIPGCASGSPGYLRVSFGGLQEVEMRAAAARLRKGIEELLHHGMVE

SEQ ID NO: 15 - protein, 394 aa, Arabidopsis lyrata
  subsp. lyrata
MGSFGMLSRRTLGTDMPVMAQIRSLMAELTNPMSLAQGVVHWQPPQKALAKVKDLVWDPMVSNYGPDEGLPELRHALLNKLREENKLTQSQVMVTAGANQAFVNLVITLCDAGDSVVMFEPYYFNSYMAFQMTGVTNIIVGPGQSDTLYPDADWLERTLSESKPTPKVVTVVNPGNPSGTYVPEPLFKRISQICKDAGCWLIVDNTYEYFIYDGLKHSCVEGDHIVNVFSFSKTYGMMGWRLGYIAYSDRLDGFATELVKIQDNIPICAAIISQRLGLYALEEGSGWITERVKSLVKNRDIVKEALEPLGKENVKGGEGAIYLWAKLPEGHRDDFKVVRWLAHRHGVVVIPGCASGGPGYLRVSFGGLQEVEMRAAAERLRKGLEELLHHGMVE

SEQ ID NO: 16 - protein, 394 aa, Ricinus communis
MGSYGMLAKRALETEMPVMVQIQELIRGAKNAISLAQGVVHWLPPKKALEKVKELVWEPRISRYGADEGIPELREALMLKLRKENKLVNSSVMVTAGANQAFVNIVLALCDPGDSVVMFAPYYFNAYMSFQMTGITNILVGPGNPKTLHPDADWLERTLSETRPIPKVVTVVNPGNPSGTYIPEPLLKRISDLCRKAGSWLIVDNTYEYFMYDGLKHSCIEGDHIVNIFSFSKAYGMMGWRVGYIAYPSGVEGFATQLLKIQDNIPICASILSQHLALYSLEVGPEWVTARVKDLVKNREILLEALSPLGEDAVRGGEGAIYLWAKLPDKYVDDFKVVRWLATRHGVVVIPGGASGCPGHVRISFGGLIEPDCRSAAERLKKGLEELVSNGMVE

SEQ ID NO: 17 - protein, 394 aa, Vitis vinifera
MSSYGMLARRVVETETPVMVQIQELIRGAGDAMSLAQGVVYWQPPKQALEKVKDLVWEPSVSRYGADDGLPELREALVEKLRQENKLYKSSVMVTAGANQAFVNLVLTLCDAGDLVVMFAPYYFNAYMSFQMTGVTNILVGPSNPKTLHPDADWLEKILLETKPAPKVVTVVNPGNPSGTYIPEPLLKRFADLCRNAGSWLVVDNTYEYFMYDGLKHTCVEGDHIVNIFSFSKAYGMMGWRVGYIAYPSNVEGLGAQLLKVQDNIPICASIFSQHLALYSLEMGPEWVTKQVKSLVINRSLIMDALSPLGKDSVKGGEGAIYLFAKLPDKYHDDFEVVRWLARKHRVVVIPGSACGTPGSLRVSFGGLREDETQIAAGRLKKGIEELVRDGMLQ

SEQ ID NO: 18 - protein, 394 aa, Vitis vinifera
MGSHPELAARALHTDLPVMVKIRELSKGTKDLMPLGQGAVYWQPPDQALEKVKEKAWEPSTSQYGTHEGLAELREALIKKLRHENELHKSSIMVTAGSNQGFLNLVLTLCDVKDSVVLFAPYFFNAYMSFQMTGITGILIGTSDPETLLPDADWLERVLSETKPAPKLVSITNPGNPTGTCIPDPLLKRISEICRRAGTWLVVDNAYEYFTYDGLKHSCVEGNHVVNLFSFSKAYGMMGWRVGYIAYPSEVQSLRTQLLKVQDNVAICASMISQSTALGCLEAGREWVRNQVKSLVRNRELLLEALSPIGQDAVKGGGGAIYIWAKLPEKYQDDFAVVCWLAKRHGVVLLPGSACGLAGCVRVTYGAIREVECEVAAKRLRAGLEELMRDGMVE

SEQ ID NO: 19 - protein, 394 aa, Populus trichocarpa
MGSYVKLAKRAVETEMPIMVQIQELVRGAKNAVSLAQGVVYWQPPKQALNKVKELVWEPSISRYGADEGIPELREALTQKLQKENKLVKSSVMVTAGANQAFVNLVLALCDAGDSVVMFAPYYFNAYMSFQMTGVTNILVGPGNPETLHPDADWLERTLSEAKPVPKVVTVVNPGNPSGTYIPDPLLKRISDLCREAGSWLIVDNTYEYFMYDGLKHSCIEGDHVVNIFSFSKAYGMMGWRVGYIAYPSGVEGFQTQLLKIQDNIPICASILSQHLALYSLEMGPEWVTEQVKDLVKNRDIILEALSPLGEGAVKGGEGAIYLWAKLPEQYVDDDKVVRWLATRHGVIVIPGGACGCPGHLRISFGGLTENDCKAAAERLRRGLEDLVSNGIVQ

SEQ ID NO: 20 - protein, 399 aa, Physcomitrella patis
MASKLARRVMGTETPVMVQMQELLRGATDVISLAQGVVHWQPPEEALSKIKIIVDEPSTSQYGADEGLPELREALLEKALLKRENKLYSSSVMVTAGANQAYVNVVLTLCDPGDRVVLFVPYYFNAFMAYQMTGVTDIVLGPSDSDLHPDADWLEKLLQNKDPKKIPKVVSIVNPGNPSGTYVPEPLLKRISEMCREAGSWLIVDNTYEYFMYDDCKHACIEGDHIINIFSFSKAHGMMGWRVGYIAYPAGVVGMGAQLLKVQDNIPICASIIGQKLALAALQVGPEWVLERVQRLGKNREVLLEALSPLGQGAVKGGEGAIYLWAQLPKDKATNEVADDVAVVRWLVKRHGVSVIPGSASGGAGFIRISYGGLTQEKCHIAAARLNKGLQELVDHGMV

SEQ ID NO: 21 - protein, 394 aa, Oryza sativa
MGSFGRLARRAVETEAPVMVKMQELLRGNKDVMSLAQGVVYWQPPEAAMNKIKEIVWEPSISKYGSDDGLPELREALLEKLRRENKLTKSSIMVTSGANQAFVNVVLTLCDAGDAVVMFAPYYFNSYMSFQMTGVTDILVGASNPETLHPDVDWLEKVLQENNPIPKLVSVVNPGNPSGAFIPKPMLERISELCRNAGAWLVVDNTYEYFMYDGMEHYCLEGNHIVNLFSFSKAYGMMGWRVGYIAHPNEADGLHAQLLKVQDNIPICASIIGQRLALYALEAGPEWIRERVRDLVKNRELLMEAMSPLGKDSVKGGEGAIYLWAKLPEKCSDDFEVVRWLANKHGVAVIPGSASGGPGYIRVSFGGLKESDTRLAAERLRRGLQELVTEGMVQ

SEQ ID NO: 22 - protein, 473 aa, Chlamydomonas reinhardtii
MSVLVSQAVAQKWKALSRRIDATDTPVIDFTRKLMASRKDAMSLAQGIVHWGPPPVALQQLATRLATEPAAVSGYVPNEGLPALRAALRTKLVERNKLHHYDVIVTPGCNQAFLNALIALCDDNDRVALFRPYYFDHLMAIQMTGGSERLVIGECDPDTMRPSLAWLERELAGPSPPKMVVLVNPCNPTGVLLSRAELDAFSAACAAAGCWLVLDNTYEDFVFAEGGEHYCPSGPQVVHLFSMSKAYGMMGWRIGYVAFPDYAHTNPDVPGEAPPSSSSSSSSNGSGQQPLTAPGALAMAQLKVQDAVCICAPAPSQALALEALGAEGRQYVDALIAPLKANRDLLRAALAPLEPHVAGGEGAIYLWARLPLGRGCEDDRAVVTWLVQQAGVCVIPGSACGMPGYIRCSYANLPPAQFPAAVSRLRAGLEHLVAHGMTAMPQQHGCWLVLDNTYEDFVFAEGGEHYCPSGPQV

SEQ ID NO: 23 - protein, 422 aa, Catharanthus roseus
MGSYGMLARRALLTDTPVMVQIQELIRGVKDCISLAQGVVYWKPPKQALEKVQQLVFEPSVSCYGADEGLPELREALIKKLQQENKLHKSSVMVTAGANQAFVNVVLTLCDAGDSVVMFAPYYFNAYMSFQMTGVTDILVGPGDPTTLHPDADWLEKTLRETKPTPKLVTVVNPGNPSGTYIPESLLKRISDICSAAGSWLVVDNTYEYFMYDGRKHVCLEDSHIVNIFSFSKAYGMMGWRVGYIAYPSEVEGLAAQLLKVQDNIPICASIISQRLALHSLEMGRDWVTDQVKDLIKNRELVLEALSPLGKDSVRGGEGAIYLWAKLPDKYHDDFEVVRWLARKHGIVIIPGSSSGCPGYVRISFGGLMEEDCQIASDRLRKGLEELVNHGMVEGSWLVVDNTYEYFMYDGRKHVCLEDSHI

SEQ ID NO: 24 - protein, 422 aa, Solanum lycopersicum
MGSFGMLARRAVLTETPVMVQIQELVRSNKGCISLAQGVVYWQPPAQALEKVKEAIWEPSVSRYGADEGLPELREALMQKLGRENNLHKSSVMVTAGANQAFVNVVLTLCDAGDSVVMFAPYYFNAHMSFQMTGVTDILVGPGDAKTLHPDADWLESTLKNTVPTPKLVTVVNPGNPSGTYIPESLLKRISDICKEAGCWLVIDNTYEYFMYDDRKHVCIEANHIVNIFSFSKAYGMMGWRVGYIAYPSEVEGLAVQLLKVQDNIPICASIISQRLALYSMEMGPEWVANQVKDLVKNREVLQEALSPLGEGAVKGGEAAIYLWAKLPDKYMDDFKVVHWLAKRHGVVLIPGSSSGCPGYLRIAFGGLIEKDCRVAAERLRKGLEELVNFGMVSGCWLVIDNTYEYFMYDDRKHVCIEANHI

SEQ ID NO: 25 - protein, 422 aa, Populus trichocarpa
MGSYVKLAKRAVETEMPIMVQIQELVRGAKNAVSLAQGVVYWQPPKQALNKVKELVWEPSISRYGADEGIPELREALTQKLQKENKLVKSSVMVTAGANQAFVNLVLALCDAGDSVVMFAPYYFNAYMSFQMTGVTNILVGPGNPETLHPDADWLERTLSEAKPVPKVVTVVNPGNPSGTYIPDPLLKRISDLCREAGSWLIVDNTYEYFMYDGLKHSCIEGDHVVNIFSFSKAYGMMGWRVGYIAYPSGVEGFQTQLLKIQDNIPICASILSQHLALYSLEMGPEWVTEQVKDLVKNRDIILEALSPLGEGAVKGGEGAIYLWAKLPEQYVDDDKVVRWLATRHGVIVIPGGACGCPGHLRISFGGLTENDCKAAAERLRRGLEDLVSNGIVQGSWLIVDNTYEYFMYDGLKHSCIEGDHV

SEQ ID NO: 26 - protein, 422 aa, Ricinus communis
MGSYGMLAKRALETEMPVMVQIQELIRGAKNAISLAQGVVHWLPPKKALEKVKELVWEPRISRYGADEGIPELREALMLKLRKENKLVNSSVMVTAGANQAFVNIVLALCDPGDSVVMFAPYYFNAYMSFQMTGITNILVGPGNPKTLHPDADWLERTLSETRPIPKVVTVVNPGNPSGTYIPEPLLKRISDLCRKAGSWLIVDNTYEYFMYDGLKHSCIEGDHIVNIFSFSKAYGMMGWRVGYIAYPSGVEGFATQLLKIQDNIPICASILSQHLALYSLEVGPEWVTARVKDLVKNREILLEALSPLGEDAVRGGEGAIYLWAKLPDKYVDDFKVVRWLATRHGVVVIPGGASGCPGHVRISFGGLIEPDCRSAAERLKKGLEELVSNGMVEGSWLIVDNTYEYFMYDGLKHSCIEGDHI

SEQ ID NO: 27 - protein, 422 aa, Gossypium hirsutum
MGSNGMLARRIVETEMPIMVQIQQLIRGAKNAVSLAQGVVYWKPPKQALDKVKALVEEPSVSCYGADEGLPELREALIRKLRQENNLQRSSVMVTAGANQAFVNLVLTLCDAGDSVVMFAPYYFNAYMSFQMTGVTKILVGPGYPKTLYPDADWLERTLLETKPVPKLVTVVNPGNPSGTYIPEPLLKRISDICKNAGCWLVVDNTYEYFMYDGLKHSCVEGDHIVNIFSFSKAYGMMGWRVGYIAYPTEVEGLATQLLKVQDNIPICASLISQQLALHSLELGPEWVREQVKDLVKNREIVIEALSPLGEGAVLGGEGAIYLWATLPEKCMDDVKVVHWLAHRHGVVVIPGSACGSPGHLRISFGGLMENDCRAAAQRLKTGLEELVKHGLVQGCWLVVDNTYEYFMYDGLKHSCVEGDHI

SEQ ID NO: 28 - protein, 422 aa, Arabidopsis thaliana
MGSFGMLSRRTLGTDMPVMAQIRSLMAELTNPMSLAQGVVHWQPPQKALEKVKELVWDPIISSYGPDEGLPELRQALLKKLREENKLTNSQVMVTAGANQAFVNLVITLCDAGDSVVMFEPYYFNSYMAFQMTGVTNIIVGPGQSDTLYPDADWLERTLSESKPTPKVVTVVNPGNPSGTYVPEPLLKRIAQICKDAGCWLIVDNTYEYFMYDGLKHCCVEGDHIVNVFSFSKTYGMMGWRLGYIAYSERLDGFATELVKIQDNIPICAAIISQRLAVYALEEGSGWITERVKSLVKNRDIVKEALEPLGKENVKGGEGAIYLWAKLPEGHRDDFKVVRWLAHRHGVVVIPGCASGSPGYLRVSFGGLQEVEMRAAAARLRKGIEELLHHGMVEGCWLIVDNTYEYFMYDGLKHCCVEGDHI

SEQ ID NO: 29 - protein, 422 aa, Zea mays
MGSFAKLARRAVETDAPVMVKIQELLRGAKDVMSLAQGVVYWQPPESAMDKIEKIIREPIVSKYGSDDGLPELREALLEKLSRENKLTKSSVMVTAGANQAFVNLVLTLCDAGDSVVMFAPYYFNAYMSFQMTGVTDILVGGCDPKTLHPDVDWLEKVLKENEPIPKLVTVVNPGNPSGAFIPRPMLERISDLCKNAGAWLVVDNTYEYFMYDGMEHYCLEDAHIVNLFSFSKAYGMMGWRVGYIAFPNEADGFHDQLLKVQDNIPICASIIGQRLALYSLEAGPEWIKERVKDLVKNRALLVEALSPLGEDNVKGGEGAIYLWAKLPDSCSDDFEAVRWLANKHGVAVIPGSASGGPGYIRVSFGGLKEEDTRLAAERLRRGLQELVTDGMVQGAWLVVDNTYEYFMYDGMEHYCLEDAHI

SEQ ID NO: 30 - protein, 422 aa, Oryza sativa
MGSFGRLARRAVETEAPVMVKMQELLRGNKDVMSLAQGVVYWQPPEAAMNKIKEIVWEPSISKYGSDDGLPELREALLEKLRRENKLTKSSIMVTSGANQAFVNVVLTLCDAGDAVVMFAPYYFNSYMSFQMTGVTDILVGASNPETLHPDVDWLEKVLQENNPIPKLVSVVNPGNPSGAFIPKPMLERISELCRNAGAWLVVDNTYEYFMYDGMEHYCLEGNHIVNLFSFSKAYGMMGWRVGYIAHPNEADGLHAQLLKVQDNIPICASIIGQRLALYALEAGPEWIRERVRDLVKNRELLMEAMSPLGKDSVKGGEGAIYLWAKLPEKCSDDFEVVRWLANKHGVAVIPGSASGGPGYIRVSFGGLKESDTRLAAERLRRGLQELVTEGMVQGAWLVVDNTYEYFMYDGMEHYCLEGNHI

SEQ ID NO: 31 - protein, 422 aa, Brachypodium distachyon
MSSFAKLAKRAVETEAPVMVKMQKLLGGLKDVMSLAQGIVYWQPPEEALNKVKEIVWEPSTSKYGSDDGLPELREALLEKLHRENKLTKSSVMVTAGANQAFVNLVLTLCDAGDSVVMFAPYYFNAYMSFQMTGVTDILVGACNSTTLHPDVDWLEKVLKGNGPIPKLVTVVNPGNPSGAFIPKPMLQRISDLCRNAGSWLVVDNTYEYFMYDGMEHYCLEDAHIINIFSFSKAHGMMGWRVGYIAYPAGVVGMHAQLLKVQDNIPICASIIGQRLALHALEAGPEWIRERVKDLVKNRELLVEAMSPLGEDAVRGGEGAIYLWAKLPDTCSDDFEAVSWLASKHGVAVIPGSACGSPGFIRISFGGLKEADTRLAAERLRRGLQELVTDGMVQGSWLVVDNTYEYFMYDGMEHYCLEDAHI

SEQ ID NO: 32 - protein, 418 aa, Selaginella moellendorfii
MAGFSRRILATDTPVMVQVQELLQGRNDVVSLAQGVVHWQPPPQALEKVKDLIQEISTSRYGPDDGLPELRLALSEKLKKENKLFKSSVMVTAGANQAYANLALSLCDPGDSVVMFAPYYFNAYTTFQMTGIYDIVVVPSHPDTCHPDPEMLEKALREHTPKPRIVNVVNPGNPSGTYLPEETLKRISELCKNAGVWLVVDNTYEYFMYDGRKHVCIEGDHILNVFSFSKAYGMMGWRIGYIAYPSTVPGFGEQLLKVQDNVPICASIIGQKLALAALDAGPQWVLERVARLGKNRAMVLDALSPLGQGAVKGGEGAIYFWAKLPEKFPDDVAVVKWLVSKHKVMVVPGSASGGKGCIRISYGGLDEGSCEIAAARLKAGLEELVLRGCVGVWLVVDNTYEYFMYDGRKHVCIEGDHI

SEQ ID NO: 33 - protein, 422 aa, Vitis vinifera
MGSHPELAARALHTDLPVMVKIRELSKGTKDLMPLGQGAVYWQPPDQALEKVKEKAWEPSTSQYGTHEGLAELREALIKKLRHENELHKSSIMVTAGSNQGFLNLVLTLCDVKDSVVLFAPYFFNAYMSFQMTGITGILIGTSDPETLLPDADWLERVLSETKPAPKLVSITNPGNPTGTCIPDPLLKRISEICRRAGTWLVVDNAYEYFTYDGLKHSCVEGNHVVNLFSFSKAYGMMGWRVGYIAYPSEVQSLRTQLLKVQDNVAICASMISQSTALGCLEAGREWVRNQVKSLVRNRELLLEALSPIGQDAVKGGGGAIYIWAKLPEKYQDDFAVVCWLAKRHGVVLLPGSACGLAGCVRVTYGAIREVECEVAAKRLRAGLEELMRDGMVEGTWLVVDNAYEYFTYDGLKHSCVEGNHV

SEQ ID NO: 34 - protein, 15 aa, Catharanthus roseus
ALLTDTPVMVQIQEL

SEQ ID NO: 35 - protein, 16 aa, Catharanthus roseus
VNIFSFSKAYGMMGWR

SEQ ID NO: 36 - protein, 16 aa, Catharanthus roseus
LKVQDNIPICASIISQ

SEQ ID NO: 37 - protein, 15 aa, Solanum lycopersicum
AVLTETPVMVQIQEL

SEQ ID NO: 38 - protein, 16 aa, Solanum lycopersicum
VNIFSFSKAYGMMGWR

SEQ ID NO: 39 - protein, 16 aa, Solanum lycopersicum
LKVQDNIPICASIISQ

SEQ ID NO: 40 - protein, 15 aa, Populus trichocarpa
AVETEMPIMVQIQEL

SEQ ID NO: 41 - protein, 16 aa, Populus trichocarpa
VNIFSFSKAYGMMGWR

SEQ ID NO: 42 - protein, 16 aa, Populus trichocarpa
LKIQDNIPICASILSQ

SEQ ID NO: 43 - protein, 15 aa, Ricinus communis
ALETEMPVMVQIQEL

SEQ ID NO: 44 - protein, 16 aa, Ricinus communis
VNIFSFSKAYGMMGWR

SEQ ID NO: 45 - protein, 16 aa, Ricinus communis
LKIQDNIPICASILSQ

SEQ ID NO: 46 - protein, 15 aa, Gossypium hirsutum
IVETEMPIMVQIQQL

SEQ ID NO: 47 - protein, 16 aa, Gossypium hirsutum
VNIFSFSKAYGMMGWR

SEQ ID NO: 48 - protein, 16 aa, Gossypium hirsutum
LKVQDNIPICASLISQ

SEQ ID NO: 49 - protein, 15 aa, Arabidopsis thaliana
TLGTDMPVMAQIRSL

SEQ ID NO: 50 - protein, 16 aa, Arabidopsis thaliana
VNVFSFSKTYGMMGWR

SEQ ID NO: 51 - protein, 16 aa, Arabidopsis thaliana
VKIQDNIPICAAIISQ

SEQ ID NO: 52 - protein, 15 aa, Zea mays
AVETDAPVMVKIQEL

SEQ ID NO: 53 - protein, 16 aa, Zea mays
VNLFSFSKAYGMMGWR

SEQ ID NO: 54 - protein, 16 aa, Zea mays
LKVQDNIPICASIIGQ

SEQ ID NO: 55 - protein, 15 aa, Oryza sativa
AVETEAPVMVKMQEL

SEQ ID NO: 56 - protein, 16 aa, Oryza sativa
VNLFSFSKAYGMMGWR

SEQ ID NO: 57 - protein, 16 aa, Oryza sativa
LKVQDNIPICASIIGQ

SEQ ID NO: 58 - protein, 15 aa, Brachypodium distachyon
AVETEAPVMVKMQKL

SEQ ID NO: 59 - protein, 16 aa, Brachypodium distachyon
VNIFSFSKAYGMMGWR

SEQ ID NO: 60 - protein, 16 aa, Brachypodium distachyon
LKVQDNIPICASIIGQ

SEQ ID NO: 61 - protein, 15 aa, Physcomitrella patens
VMGTETPVMVQMQEL

SEQ ID NO: 62 - protein, 16 aa, Physcomitrella patens
INIFSFSKAHGMMGWR

SEQ ID NO: 63 - protein, 16 aa, Physcomitrella patens
LKVQDNIPICASIIGQ

SEQ ID NO: 64 - protein, 15 aa, Selaginella moellendorfii
ILATDTPVMVQVQEL

SEQ ID NO: 65 - protein, 16 aa, Selaginella moellendorfii
LNVFSFSKAYGMMGWR

SEQ ID NO: 66 - protein, 16 aa, Selaginella moellendorfii
LKVQDNVPICASIIGQ

SEQ ID NO: 67 - protein, 15 aa, Vitis vinifera
ALHTDLPVMVKIREL

SEQ ID NO: 68 - protein, 16 aa, Vitis vinifera
VNLFSFSKAYGMMGWR

SEQ ID NO: 69 - protein, 16 aa, Vitis vinifera
LKVQDNVAICASMISQ

SEQ ID NO: 70 - protein, 15 aa, Chlamydomonas reinhardtii
IDATDTPVIDFTRKL

SEQ ID NO: 71 - protein, 16 aa, Chlamydomonas reinhardtii
VHLFSMSKAYGMMGWR

SEQ ID NO: 72 - protein, 16 aa, Chlamydomonas reinhardtii
LKVQDAVCICAPAPSQ